SEQUENCE LISTING (29 sequences)

SEQ ID NO: 1 - protein, 267 aa, Homo sapiens CD82 [sequence omitted]

SEQ ID NO: 2 - protein, 266 aa, Mus musculus CD82
MGAGCVKVTKYFLFLFNLLFFILGAVILGFGVWILADKNSFISVLQTSSSSLQVGAYVFIGVGAITIVMGFLGCIGAVNEVRCLLGLYFVFLLLILIAQVTVGVLFYFNADKLKKEMGNTVMDIIRNYTANATSSREEAWDYVQAQVKCCGWVSHYNWTENEELMGFTKTTYPCSCEKIKEEDNQLIVKKGFCEADNSTVSENNPEDWPVNTEGCMEKAQAWLQENFGILLGVCAGVAVIELLGLFLSICLCRYIHSEDYSKVPKY

SEQ ID NO: 3 - protein, 338 aa, Homo sapiens DARC
MASSGYVLQAELSPSTENSSQLDFEDVWNSSYGVNDSFPDGDYGANLEAAAPCHSCNLLDDSALPFFILTSVLGILASSTVLFMLFRPLFRWQLCPGWPVLAQLAVGSALFSIVVPVLAPGLGSTRSSALCSLGYCVWYGSAFAQALLLGCHASLGHRLGAGQVPGLTLGLTVGIWGVAALLTLPVTLASGASGGLCTLIYSTELKALQATHTVACLAIFVLLPLGLFGAKGLKKALGMGPGPWMNILWAWFIFWWPHGVVLGLDFLVRSKLLLLSTCLAQQALDLLLNLAEALAILHCVATPLLLALFCHQATRTLLPSLPLPEGWSSHLDTLGSKS

SEQ ID NO: 4 - protein, 334 aa, Mus musculus DARC
MGNCLYPVENLSLDKNGTQFTFDSWNYSFEDNYSYELSSDYSLTPAAPCYSCNLLGRSSLPFFMLTSVLGMLASGGILFAILRPFFHWQICPSWPILAELAVGSALFSIAVPILAPGLHSAHSTALCNLGYWVWYTSAFAQALLIGCYACLNPRLNIGQLRGFTLGLSVGLWGAAALLGLPVALASDAYNGFCAFPSSRDMEALKYMHYAICFTIFTVLPPTLLAAKGLKIALSKGPGPWVSVLWIWFIFWWPHGMVLIFDALVRSKIVLLYTCQSQKILDAMLNVTEALSMLHCVATPLLLALFCHQTTRRSLSSLSLPTRQASQMDALAGKS

SEQ ID NO: 5 - DNA, 23 bp, Artificial Sequence
  CD9 forward primer
agtgcatcaaatacctgctcttc

SEQ ID NO: 6 - DNA, 23 bp, Artificial Sequence
  CD9 reverse primer
ctttaatcacctcatccttgtgg

SEQ ID NO: 7 - DNA, 23 bp, Artificial Sequence
  CD37 forward primer
cttcgttttcaacctcttcttct

SEQ ID NO: 8 - DNA, 23 bp, Artificial Sequence
  CD37 reverse primer
aactgtgcatagtcccaactctc

SEQ ID NO: 9 - DNA, 23 bp, Artificial Sequence
  CD81 forward primer
ttctacgtgggcatctacattct

SEQ ID NO: 10 - DNA, 23 bp, Artificial Sequence
  CD81 reverse primer
gctgttcctcagtatggtggtag

SEQ ID NO: 11 - DNA, 23 bp, Artificial Sequence
  CD151 forward primer
tgcctcaagtacctgctctttac

SEQ ID NO: 12 - DNA, 23 bp, Artificial Sequence
  CD151 reverse primer
ctgactggtggtatctcttgacc

SEQ ID NO: 13 - DNA, 24 bp, Artificial Sequence
  KAI1(CD82) forward primer type I
cactacaactggacagagaacgag

SEQ ID NO: 14 - DNA, 25 bp, Artificial Sequence
  KAI1(CD82) reverse primer type I
tgtagtcttcagaatgaatgtaccg

SEQ ID NO: 15 - DNA, 20 bp, Artificial Sequence
  Primer A
gggtcccctaggaaattcaa

SEQ ID NO: 16 - DNA, 25 bp, Artificial Sequence
  Primer B
atgatgcagatgttctctcagggtg

SEQ ID NO: 17 - DNA, 22 bp, Artificial Sequence
  Primer C
acaggggactcaccctacaagg

SEQ ID NO: 18 - DNA, 23 bp, Artificial Sequence
  P21 forward primer
gagaacggtggaactttgacttc

SEQ ID NO: 19 - DNA, 24 bp, Artificial Sequence
  P21 reverse primer
gtgatagaaatctgtcaggctggt

SEQ ID NO: 20 - DNA, 22 bp, Artificial Sequence
  P27 forward primer
catgaagaactaacccgggact

SEQ ID NO: 21 - DNA, 22 bp, Artificial Sequence
  P27 reverse primer
cagagtttgcctgagacccaat

SEQ ID NO: 22 - DNA, 23 bp, Artificial Sequence
  P57 forward primer
agatctgacctcagacccaattc

SEQ ID NO: 23 - DNA, 22 bp, Artificial Sequence
  P57 reverse primer
gctcttgattctcgtcctgctc

SEQ ID NO: 24 - DNA, 18 bp, Artificial Sequence
  KAI1(CD82) forward primer type II
gcctgggactacgtgcag

SEQ ID NO: 25 - DNA, 21 bp, Artificial Sequence
  KAI1(CD82) reverse primer type II
cctcgttctctgtccagttgt

SEQ ID NO: 26 - DNA, 25 bp, Artificial Sequence
  TGF-beta1 forward primer
cggactactatgctaaagaggtcac

SEQ ID NO: 27 - DNA, 26 bp, Artificial Sequence
  TGF-beta1 reverse primer
gagtttgttatctttgctgtcacaag

SEQ ID NO: 28 - DNA, 23 bp, Artificial Sequence
  TGF-beta R2 forward primer
catcttctactgctaccgtgtcc

SEQ ID NO: 29 - DNA, 23 bp, Artificial Sequence
  TGF-beta R2 reverse primer
atgttctcatgcttcaggttgat

(19) United States
(12) Patent Application Publication
(10) Pub. No.: US 2018/0133257 A1
(43) Pub. Date: May 17, 2018

(54) KAI1 PROTEIN CONTROLLING CELL CYCLE OF HEMATOPOIETIC STEM CELL, AND USE THEREOF

(71) Applicant: SEOUL NATIONAL UNIVERSITY R & DB FOUNDATION, Seoul (KR)

(72) Inventors: Hyo-Soo KIM, Seoul (KR); Jin HUR, Seoul (KR); Jae Il CHOI, Seoul (KR)

(73) Assignee: SEOUL NATIONAL UNIVERSITY R & DB FOUNDATION, Seoul (KR)

(21) Appl. No.: 15/572,106

(22) PCT Filed: May 4, 2016
(86) PCT No.: PCT/KR2016/004707

§ 371 Date: Nov. 6, 2017

(30) Foreign Application Priority Data
May 6, 2015 (KR) 10-2015-0063203

(51) Int. Cl. A61K 35/28; C07K 14/705; A61K 38/17; A61P 35/00; C12N 5/00; C12N 5/078

(52) U.S. Cl. CPC A61K 35/28; C07K 14/705; A61K 38/17; A61P 35/00; A61K 2035/124; C12N 5/0634; C12N 2502/1171; C12N 2502/1157; C12N 5/0081

(57) ABSTRACT

Provided is a KAI1 protein controlling the cell cycle of a hematopoietic stem cell and a use thereof, and more specifically, a composition including a KAI1(CD82) polypeptide or a gene encoding the same for controlling the cell cycle of a hematopoietic stem cell, or a pharmaceutical composition for preventing or treating blood tumors. Further provided, KAI1(CD82) is expressed only in a hematopoietic stem cell (LT-HSC) which is the uppermost stem cell in the hierarchy of the hematopoietic stem cell, can obtain an LT-HSC with high purity using the KAI1, and thus can inhibit various side effects of hematopoietic stem cell transplantation by transplanting only an LT-HSC with high purity. In addition, KAI1 importantly acts on the quiescence of an LT-HSC, and has resistance against cell damage due to various stresses (5-FU, irradiation, etc.) by maintaining the quiescence of the LT-HSC. Thus, it is expected that the cell can be used for making a cell bank and can be used as a blood tumor cell therapy product through cell transplantation.

### Statement Regarding Government Rights

The present invention was undertaken with the support of “Studying regulation mechanisms of retinal angiogenesis and developing an innovative therapy for wet age-related macular degeneration using membrane protein K” No. 2017R1A2B2012305 grant funded by Basic Science Research Program through the National Research Foundation of Korea (NRF).

### CROSS-REFERENCE TO RELATED APPLICATION

This application claims priority to and the benefit of Korean Patent Application No. 10-2015-0063203 filed on May 6, 2015 and International Patent Application No. PCT/KR2016/004707, filed on May 4, 2016, the disclosure of which is incorporated herein by reference in its entirety.

### SEQUENCE LISTING

The Sequence Listing submitted in text format (.txt) filed on Nov. 6, 2017, named “SequenceListing.txt”, created on Nov. 6, 2017 (20.9 KB), is incorporated herein by reference.

### TECHNICAL FIELD

The present invention relates to a KAI1 protein regulating the cell cycle of hematopoietic stem cells and a use thereof, and more particularly, to a composition for regulating the cell cycle of hematopoietic stem cells including a KAI1 (CD82) polypeptide or a gene encoding the polypeptide, and a pharmaceutical composition for mass culture and maintenance/storage of stem cells or preventing or treating blood tumor.

### BACKGROUND ART

Leukemia is a type of blood tumor in which blood cells, particularly white blood cells, are abnormally grown, and therefore a large amount of improperly grown white blood cells are contained in the blood (in addition, such white blood cells including their cell organelles are much larger than normal white blood cells in size in biopsy). Compared to abnormally proliferated white blood cells, the number of normal blood cells is ultimately decreased, and therefore basic blood functions such as oxygen delivery and nutrient supply as well as immune functions cannot be performed. In addition, abnormal white blood cells have a reaction like that of an autoimmune disease, and may destroy normal tissue. Leukemia is divided into acute and chronic forms according to the degree of cell differentiation, and also divided into myelogenous leukemia and lymphocytic leukemia according to the origin of cells. Consequently, leukemia is subdivided into four large groups: i) acute myelogenous leukemia (AML); ii) chronic myelogenous leukemia (CML); iii) acute lymphocytic leukemia (ALL); and iv) chronic lymphocytic leukemia (CLL).

One method for treating leukemia is hematopoietic stem cell transplantation. Hematopoietic stem cells or hemocytoblasts are cells capable of potentially differentiating into the major components of blood. The hematopoietic stem cells can differentiate into cells produced from the bone marrow cells (monocytes, macrophages, neutrocytes, basophilic leukocytes, red blood cells, thrombocytes, etc.), lymphocytic cells (T cells, B cells, and NK cells), etc. These cells account for a relatively small proportion, making up 1/10,000 of the cells in bone marrow tissue, but have self-replicating capability and therefore can always produce the components of blood at the right time. For this reason, when an abnormality occurs in cell differentiation like leukemia, or the number of hematopoietic stem cells is reduced like aplastic anemia, these diseases may be fundamentally treated by transplantation of hematopoietic stem cells.

However, after hematopoietic stem cell transplantation, due to a long-term decrease in immune function, repeated bacterial, viral, or fungal infection may occur, and recovery of immune function may depend on the type of hematopoietic stem cell graft, the duration of immunosuppressant administration, or a graft-versus-host disease. In the case of an allograft, a graft-versus-host disease may occur and become a major factor in determining the outcome of transplantation as well as recurrence.

Therefore, minimizing such hematopoietic stem cell transplantation has become a major subject of study, but sufficient results still have not been produced.

### DISCLOSURE

### [Technical Problem]

To solve the above-mentioned problems, the present invention is directed to providing a composition for regulating the cell cycle of hematopoietic stem cells, which includes a KAI1(CD82) polypeptide or a gene encoding the polypeptide, or a pharmaceutical composition for preventing or treating blood tumor.

The present invention is also directed to providing a method for purifying hematopoietic stem cells having a resistance to stress, which includes the following steps:

(a) analyzing KAI1 expression in an isolated hematopoietic stem cell population; and

(b) isolating and harvesting only KAI1-expressed hematopoietic stem cells. However, technical problems to be solved in the present invention are not limited to the above-described problems, and other problems which are not described herein will be fully understood by those of ordinary skill in the art from the following description.

### [Technical Solution]

To achieve the objects of the present invention, the present invention provides a composition for regulating the cell cycle of hematopoietic stem cells, which includes a KAI1(CD82) polypeptide or a gene encoding the polypeptide.

In one exemplary embodiment of the present invention, the composition may further include a DARC polypeptide or a gene encoding the same.

In another exemplary embodiment of the present invention, the KAI1 polypeptide may consist of amino acids of SEQ ID NO: 1 or SEQ ID NO: 2.

In still another exemplary embodiment of the present invention, the DARC polypeptide may consist of amino acids of SEQ ID NO: 3 or SEQ ID NO: 4.

In yet another exemplary embodiment of the present invention, the hematopoietic stem cells may be long-term hematopoietic stem cells (LT-HSCs), short-term hematopoietic stem cells (ST-HSCs), or a multipotent progenitors (MPPs).

In yet another exemplary embodiment of the present invention, the composition may maintain the G0 phase of the hematopoietic stem cells.

In yet another exemplary embodiment of the present invention, the composition may increase TGF-β1 secretion via PKC.

The present invention provides a pharmaceutical composition for preventing or treating blood tumor, which includes a KAI1(CD82) polypeptide or a gene encoding the same.

In one exemplary embodiment of the present invention, the composition may further include a DARC polypeptide or a gene encoding the same.

In another exemplary embodiment of the present invention, the blood tumor may be selected from the group consisting of lymphoma, multiple myeloma, myelogenous leukemia, and lymphocytic leukemia.

The present invention provides a method for purifying hematopoietic stem cells having a resistance to stress, which includes the following steps:

(a) analyzing KAI1 expression in an isolated hematopoietic stem cell population; and

(b) isolating and harvesting only KAI1-expressed hematopoietic stem cells.

The present invention provides a cellular therapy product for treating blood tumor, which includes hematopoietic stem cells purified by the above-described method.

The present invention provides a method for harvesting quiescent hematopoietic stem cells, which includes increasing expression of KAI1 in hematopoietic stem cells, wherein the hematopoietic stem cells are LT-HSCs, ST-HSCs, or MPPs.

In one exemplary embodiment of the present invention, the hematopoietic stem cells may be cultured with a rhDARC, DARC positive monocytes, DARC positive macrophages, or a DARC positive macrophage culture solution to increase or maintain KAI1 expression.

The present invention provides quiescent hematopoietic stem cells harvested by the above-described method.

In one exemplary embodiment of the present invention, the quiescent hematopoietic stem cells may be enhanced in in vivo transplantation efficiency, proliferation, differentiation, storage ability, stability.

In another exemplary embodiment of the present invention, the quiescent hematopoietic stem cells may improve organ transplantation and/or regeneration ability.

The present invention provides a method for preventing or treating blood tumor, which includes administering a pharmaceutically acceptable amount of the pharmaceutical composition to a subject.

The present invention provides a use of the pharmaceutical composition to prevent or treat blood tumor.

### [Advantageous Effects]

According to the present invention, since KAI1 (CD82) can be expressed in the uppermost hematopoietic stem cells (LT-HSCs) of the hierarchy of hematopoietic stem cells and LT-HSCs can be obtained with high purity using the KAI1, various side effects of hematopoietic stem cell transplantation can be inhibited by transplanting only LT-HSCs with high purity.

Also, KAI1 significantly acts on the quiescence of LT-HSCs to sort quiescent LT-HSCs with high purity, and therefore will be a beneficial tool for studying quiescent LT-HSCs from now.

Moreover, KAI1 significantly acts on the quiescence of LT-HSCs and maintains the quiescence of LT-HSCs to have a resistance to cell damage caused by various types of stress (5-FU, irradiation, etc.). Therefore, KAI1 is expected to form a cell bank using the LT-HSCs and expected to be used as a cellular therapy product for blood tumor through cell transplantation.

### DESCRIPTION OF DRAWINGS

FIG. 1 shows the result of sorting each population of LT-HSC, ST-HSC, and MPP cells.

FIG. 2 shows the result of determining expression levels of CD9, CD37, KAI1 (CD82), and CD151 using RT-PCR by isolating RNA from LT-HSCs, ST-HSCs, and MPPs.

FIG. 3 shows the result of identifying the expression of KAI1, CD9, CD37 and CD151 by staining LT-HSCs, ST-HSCs and MPPs.

FIG. 4 shows the result of additionally staining KAI1 using markers (HSC1, MPP1, MPP2), which has been reported in a recent research paper.

FIGS. 5 and 6 show the result of staining a bone of a C57BL/6 mouse to determine a location of KAI1 (+)LT-HSCs in the bone.

FIGS. 7 and 8 show the result of confirming KAI1 Knock-out mouse Genomic DNA manufactured in the present invention.

FIGS. 9 to 11 show the result of comparing LT-HSC, ST-HSC, and MPP cell count in bone marrow (BM) between a wild type (WT) and KAI1 K/O mice using FACS analysis.

FIG. 12 shows the result of a colony forming unit (CFU) assay for BM cells of WT and KAI1 K/O mice using MethoCult™ GF M343.

FIG. 13 shows the result of a long-term culture-initiating cell (LTC-IC) assay to quantify LT-HSCs.

FIGS. 14 and 15 show the results of ki67 and Hoechst 33342 analyses for LT-HSCs (CD34-LSK) using LSR II after bone marrow (BM) was obtained from WT and KAI1 K/O mice.

FIGS. 16 and 17 show the results of confirming incorporation of exogenous BrdU into Genomic DNA to determine proliferative activity in WT and KAI1 K/O mice in vivo.

FIG. 18 shows the results of confirming expression of p21, p27, and p57 in LT-HSCs of WT and KAI1 K/O mice using RT-PCR.

FIG. 19 shows the result of confirming Rb phosphorylation in LT-HSCs of WT and KAI1 K/O mice using FACS analysis.

FIG. 20 shows the result of comparing gene expression of KAI1 in Lin(−)CD34(−) cells and Lin(−)CD34(+) cells.

FIG. 21 shows the result of confirming KAI1 knock-down by qRT-PCR after KAI1 knock-down is induced in EML cells using a shRNA construct.

FIG. 22 shows the result of confirming the increase in KAI1 expression by qRT-PCR after KAI1 is over-expressed in EML cells.

FIGS. 23 and 24 shows the results of RNA sequencing analysis for KAI1 knock-down and KAI1 overexpression EML cells.

FIGS. 25 and 26 show the results of confirming the increase/decrease in TGF-β1 and TGFR2 expression in KAI1 knock-down and KAI1 overexpression EML cells using RT-PCR and Western blotting.

FIGS. 27 and 28 show the result of comparing TGF-β1 and TGFR2-positive cells in LT-HSCs of WT and KAI1 K/O mice.

FIGS. 29 and 30 show the results of determining TGF-β1 expression and secretion using Western blotting after KAI1 overexpression EML cells are treated with an inhibitor.

FIG. 31 shows the result of comparing phosphorylation of Smad2 and Smand3 as downstream pathways of TGF-β1/TGFR and the increase/decrease in expression of CDK inhibitors such as p21, p27, and p57 in KAI1 knock-down and KAI1 overexpression EML cells using Western blotting.

FIG. 32 shows the result of determining the increase/decrease in p21, p27, and p57 expression using Western blotting after KAI1 overexpression EML cells are treated with a TGFR inhibitor or a neutral antibody against TGF-β1.

FIGS. 33 and 34 show the results of comparing the cell cycles of Mock/, KAI1 O/E EML using FACS.

FIGS. 35 to 38 show continuous changes in KAI1-positive LT HSCs and proliferating blood cells in BM by injecting 5-FU into WT mice, following sub-lethal irradiation of the WT mice.

FIGS. 39 and 40 show the results of recovery of total BM or LSK cells in BM cells of KAI1^{-/-} and WT mice following sub-lethal irradiation.

FIG. 41 shows a schematic diagram illustrating a competitive BM transplantation (BMT) experiment to examine whether KAI1 affects long term repopulating capacity of HSCs.

FIG. 42 shows the result of FACS analysis of BM cells obtained according to the process illustrated in FIG. 41.

FIGS. 43 and 44 show the results of comparing LSK and LT-HSCs in BM cells of mice receiving HSPCs of KAI1^{-/-} mice and BM cells of mice receiving HSPCs of WT mice at the second BMT in serial BMT.

FIGS. 45 and 46 show the results of comparing cell cycles of CD34- LSK cells in mice receiving HSPCs derived from KAI1^{-/-} mice and mice receiving HSPCs derived from WT.

FIG. 47 shows the result of the Western blotting of a duffy antigen receptor for chemokines (DARC) following immunoprecipitation in EML cells using a KAI1 antibody.

FIG. 48 shows the result of identifying F4/80(+) cells or Lin(−) cells using a magnetic-activated cell sorting (MACS) technique after being co-cultured and then stained with KAI1 and DARC.

FIGS. 49A and 49B show the results of fluorescence staining after paraffin blocks of a femur of a C57 mouse (FIG. 49A) or a femur of a Tie2-GFP mouse (FIG. 49B) are prepared, sectioned, and then subjected to deparaffinization and a retrieval process to confirm in vivo interaction between KAI1 and DARC.

FIG. 50 shows the result of confirming DARC expression in endothelial cells, stromal cells, and monocytes/macrophages to identify the type of DARC(+) cells.

FIG. 51 shows the result of comparing degrees of KAI1 expression in Lin(−)CD34(−) and Lin(−)CD34(+).

FIG. 52 shows the result of confirming PKC phosphorylation caused by KAI1 activity.

FIG. 53 shows the result of confirming that rhDARC improves PKCa phosphorylation by KAI1.

FIG. 54 shows the result of analyzing cell cycles after qEML cells are co-cultured with Mock Raw 264.7 cells, DARC Knock-down Raw 264.7 cells, and DARC Knock-down Raw 264.7 cells, pretreated with rhDARC, and cultured for 2 days.

FIG. 55 shows the result of confirming KAI1(+) cell population after being sorted by MACS using Lin(−)CD34(−) cells and then cultured with a stem cell factor (SCF).

FIGS. 56 and 57 show the results of confirming DARC expression in Raw 264.7 cells in stress or mobilization niche such as G-CSF or 5-FU.

FIG. 58 shows the result of confirming, following reduction of DARC expression in Raw 264.7 cells using shRNA, KAI1 expression when Mock Raw264.7 and DARC Knock-down Raw264.7 cells are co-cultured and SCF is added to quiescent EML cells sorted from the total EML cells.

FIG. 59 shows the result of confirming KAI1 expression when Mock and DARC Knock-down Raw 264.7 supernatants were co-cultured with EML cells to examine a paracrine effect.

FIG. 60 shows the result of confirming KAI1 expression after qEML cells are sorted from EML cells and cultured with SCF.

FIG. 61 shows the result of Western blotting for mono/poly-ubiquitin after EML cells are cultured with SCF, and 8 hours later, subjected to KAI1 immunoprecipitation (IP).

FIGS. 62 to 64 show the results of confirming KAI1-biotin conjugation after all groups of qEML only, qEML +Mock Raw 264.7 cells, qEML +DARC Knock-down Raw 264.7 cells, qEML +DARC Knock-down Raw 264.7 +rhDARC cells, and MG-132-pretreated qEML cells were cultured with SCF for 3 hours, membrane-binding antibodies are eliminated with acidic buffer (50 mM Glycine, 100 mM NaCl, pH 2.5), and endocytosis is induced.

FIGS. 65 and 66 show the result of FACS analysis for human LT-HSC after being stained with LIN, CD38, CD34, CD93, and CD45RA antibodies.

FIG. 67 shows the result of fluorescence staining after MNCs are obtained from human umbilical cord blood, and then Lin(+) CD235(+)CD235(+) cells are eliminated by MACS (first step), and KAI1(+)LT-HSC and KAI1(−)LT-HSC are sorted by FACS, subjected to starvation for 3 hours, treated with rhDARC, and subjected to fixation/permeabilization.

FIG. 68 shows the result of analyzing cell cycles after MNCs are obtained, Lin(+)CD235(+)DARC(+) cells are eliminated, subjected to starvation for 3 hours, and then treated with rhDARC for 2 days to examine whether quiescence of KAI1(+)LT-HSC is increased due to rhDARC.

FIG. 69 shows the result of confirming DARC expression in CD14(+) cells of human umbilical cord blood.

FIG. 70 shows the result of obtaining Lin(−)CD235(−)DARC(−) cells from umbilical cord blood, subjecting some of the same umbilical cord blood to MACS to sort un-touched monocytes, and then culturing [SCF, Flt3-ligand, TPO, IL-3] HSCs only by using a growth factor.

FIG. 71 shows the summary of the results of the examples.

### MODES OF THE INVENTION

As a result of research on a method for reducing side effects of hematopoietic stem cell transplantation, the inventors first found a marker which is called KAI1(CD82) expressed only in the uppermost LT-HSCs of the hierarchy of hematopoietic stem cells. In addition, it was confirmed that KAI1 significantly acts on the quiescence of LT-HSCs and maintains the quiescence of LT-HSCs, thereby having a resistance to various types of stress (5-FU, irradiation, etc.). Based on this, the present invention was achieved.

Hereinafter, the present invention will be described in detail.

The present invention provides a composition for regulating the cell cycle of hematopoietic stem cells, which includes a KAI1(CD82) polypeptide or a gene encoding the same, wherein the hematopoietic stem cells are preferably LT-HSCs, ST-HSCs or MPPs. In addition, the composition of the present invention may further include a DARC polypeptide or a gene encoding the same.

The present invention may regulate the cell cycle of the LT-HSCs by regulating the expression of the KAI1(CD82) protein which is only specifically expressed in the LT-HSCs. That is, as the expression of the KAI1(CD82) protein is increased, the G0 phase (quiescence) of the LT-HSCs may be maintained due to the increase in TGF-β1 secretion via PKC, and as expression of the KAI1(CD82) protein is decreased, proliferation and differentiation of the LT-HSCs may be promoted via a reverse signaling mechanism.

In the present invention, the KAI1(CD82) polypeptide may consist of a human-derived amino acid sequence of SEQ ID NO: 1 or a mouse-derived amino acid sequence of SEQ ID NO: 2, but the present invention is not limited thereto. The KAI1(CD82) polypeptide may include a protein represented by an amino acid sequence having 70% or more, preferably 80% or more, more preferably 90% or more, and most preferably 95% or more homology with the amino acid sequence. In addition, in the present invention, the gene may be any gene capable of encoding a KAI1 polypeptide having an amino acid sequence of SEQ ID NO: 1 or SEQ ID NO: 2.

In one exemplary embodiment of the present invention, it was confirmed that KAI1(CD82) is specifically expressed only in LT-HSCs of LT-HSCs, ST-HSCs and MPPs (refer to Example 1). In addition, as a result of confirming that the cell cycle of the LT-HSCs is regulated according to KAI1(CD82) expression, compared to WT, in LT-HSCs of a KAI1 K/O mouse, G0 phase was reduced whereas G1/S/G2 phases were extended, and the number of LT-HSCs was also decreased (refer to Examples 2 and 3). In addition, it was confirmed that KAI1 increases expression and secretion of TGF-β1 via PKC, not Erk (refer to Example 4).

From the above-mentioned experimental results, it can be seen that the cell cycle and differentiation of hematopoietic stem cells, particularly LT-HSCs, may be regulated by regulating KAI1 expression, which is effective against blood tumor. Therefore, a material for improving KAI1 or KAI1 expression may maintain the quiescence of hematopoietic stem cells may also be effective in treating blood tumor.

Therefore, in another aspect of the present invention, the present invention provides a pharmaceutical composition for preventing or treating blood tumor, which includes a KAI1(CD82) polypeptide or a gene encoding the same, and may further include a DARC polypeptide or a gene encoding the same.

The term “prevention” used herein refers to all actions of inhibiting the development of blood tumor or delaying the onset of the blood tumor by administration of the pharmaceutical composition according to the present invention.

The term “treatment” used herein refers to all actions involved in alleviating or beneficially changing symptoms of blood tumor by administration of the pharmaceutical composition according to the present invention.

The term “blood tumor,” which is a disease to be ameliorated, prevented, or treated by the composition of the present invention, may be a malignant tumor occurring in white blood cells or a lymph system, and selected from the group consisting of lymphoma, multiple myeloma, myelogenous leukemia, and lymphocytic leukemia, but the present invention is not limited thereto.

The pharmaceutical composition of the present invention may further include a pharmaceutically acceptable carrier. The pharmaceutically acceptable carrier may include saline, polyethyleneglycol, ethanol, vegetable oil, isopropylmyristate, etc., but the present invention is not limited thereto.

The composition of the present invention, which includes a KAI1 polypeptide or gene encoding the same as an active ingredient, may include the active ingredient at 0.0001 to 50 wt % with respect to the total weight.

A preferable dosage of the pharmaceutical composition of the present invention may be determined by one of ordinary skill in the art according to a condition and body weight of an individual, severity of a disease, a drug form, an administration route, and duration. However, the pharmaceutical composition of the present invention is preferably administered at 0.001 to 100 mg/kg, and more preferably 0.01 to 30 mg/kg a day. The pharmaceutical composition of the present invention may be administered once or several times a day.

The pharmaceutical composition of the present invention may be administered into a mammal such as a rat, a mouse, a stock, or a human via various routes. All methods of administration may be expected, and the pharmaceutical composition of the present invention may be administered, for example, orally, or by rectal, intravenous, intramuscular, subcutaneous, epidural, or intracerebroventricular injection.

The pharmaceutical composition of the present invention may be prepared in various pharmaceutical forms, and thus there is no limit to a drug form.

In still another aspect of the present invention, the present invention provides a method for preventing or treating blood tumor by administering a pharmaceutically effective amount of the pharmaceutical composition into a subject. The term “subject” refers to a target to be treated, and more specifically, a mammal such as a human, a non-human primate, a mouse, a rat, a dog, a cat, a horse, and a cow. In addition, it is apparent to those of ordinary skill in the art that the “pharmaceutically effective amount” used herein may be controlled in various ranges according to a body weight, age, sex, or health condition of a patient, a diet, an administration time, an administration route, an excretion rate, and a severity of a disease.

In addition, according to the present invention, the quiescence of LT-HSCs may be maintained by the expression of KAI1 (CD82), and thereby the LT-HSCs may have a resistance to various types of stress.

In another exemplary embodiment of the present invention, it was confirmed that a KAI1(CD82)-expressed mouse group has a resistance to 5-FU and irradiation (refer to Example 5).

Therefore, in yet another aspect of the present invention, the present invention provides a method for purifying hematopoietic stem cells having a resistance to stress, which includes the following steps:

(a) analyzing KAI1 expression in an isolated hematopoietic stem cell population; and

(b) isolating and collecting KAI1-expressed hematopoietic stem cells.

Meanwhile, in yet another exemplary embodiment of the present invention, as a result of examining a mechanism for regulating a ligand associated with KAI1 activity and KAI1 expression, it was confirmed that KAI1 interacts with DARC and expression is regulated by co-culture with rhDARC, DARC positive monocytes, or DARC positive macrophages (refer to Examples 7 and 8).

Therefore, in yet another aspect of the present invention, the present invention provides a method for collecting quiescent hematopoietic stem cells, which includes increasing KAI1 expression in hematopoietic stem cells, wherein the hematopoietic stem cells are LT-HSCs, ST-HSCs, or MPPs. Here, the KAI1 expression may be increased or maintained by culturing the hematopoietic stem cells with a rhDARC, DARC positive monocyte, DARC positive macrophage, or DARC positive macrophage culture solution.

In the present invention, the DARC polypeptide may consist of a human-derived amino acid sequence of SEQ ID NO: 3 or a mouse-derived amino acid sequence of SEQ ID NO: 4, but the present invention is not limited thereto. The DARC polypeptide may include a protein represented by an amino acid sequence having 70% or more, preferably 80% or more, more preferably 90% or more, and most preferably 90% or more homology with the amino acid sequence.

In yet another aspect of the present invention, the present invention provides quiescent hematopoietic stem cells collected by the above-described method. The quiescent hematopoietic stem cells of the present invention may increase in vivo transplantation efficiency, proliferation ability, differentiation ability, or stability, and may improve pancreatic transplantation, organ transplantation such as liver regeneration, or regeneration ability.

Hereinafter, to help in understanding the present invention, exemplary embodiments will be disclosed. However, the following examples are merely provided to more easily understand the present invention, and the scope of the present invention is not limited to the examples.

### EXAMPLES

### Example 1

### Identification of KAI1(CD82) as Maker of LT-HSCs

To examine whether KAI1(CD82) becomes a marker of the uppermost hematopoietic stem cells (LT-HSCs) in the hierarchy of hematopoietic stem cells, an experiment was carried out as follows:

### 1-1. Collection of Cells

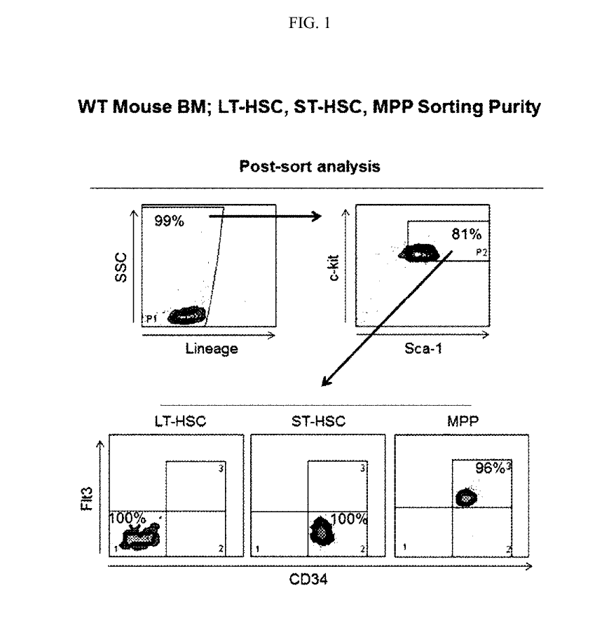

First, to obtain LT-HSCs, ST-HSCs, or MPPs, BM cells were obtained from femurs and tibias of C57BL/6 mice, and then peripheral blood mononuclear cells (PBMCs) were isolated therefrom using Histopaque-1083 (Sigma-Aldrich). In addition, the isolated PBMCs were stained using lineage cells (CD3e, CD11b, CD45R/B220, Erythroid Cells, Ly-6G, and Ly-6C), and c-kit, Sca-1, Flt3, CD34 antibodies. With well-known conventional markers, the LT-HSCs were designated as Flt3-CD34-LSK, the ST-HSCs were designated as Flt3-CD34+LSK, and MPPs were designated as Flt3+CD34+LSK, wherein the LSK indicates Lineage(−)Sca-1(+)c-kit(+). Each population was determined using an FACS Arial flow cytometer, and consequently, as shown in FIG. 1, it can be confirmed that each population was sorted with high purity.

### 1-2. Confirmation of KAI1(CD82) Expression in LT-HSCs

To confirm whether KAI1(CD82) was only expressed in the LT-HSCs, first, RNA of the cells obtained in Example 1-1 was isolated to determine expression levels of CD9, CD37, KAI1(CD82), and CD151, which are known to be tetraspanin, using RT-PCR. Primer information used herein is shown in Table 1.

TABLE 1

| Type | Primer sequence | |
|---|---|---|
| CD9 | FW (Forward) | 5′-AGTGCATCAAATACCTGCTCTTC-3′ (SEQ ID NO: 5) |
| | RV (Reverse) | 5′-CTTTAATCACCTCATCCTTGTGG-3′ (SEQ ID NO: 6) |
| CD37 | FW (Forward) | 5′-CTTCGTTTTCAACCTCTTCTTCT-3′ (SEQ ID NO: 7) |
| | RV (Reverse) | 5′-AACTGTGCATAGTCCCAACTCTC-3′ (SEQ ID NO: 8) |
| CD81 | FW (Forward) | 5′-TTCTACGTGGGCATCTACATTCT-3′ (SEQ ID NO: 9) |
| | RV (Reverse) | 5′-GCTGTTCCTCAGTATGGTGGTAG-3′ (SEQ ID NO: 10) |
| CD151 | FW (Forward) | 5′-TGCCTCAAGTACCTGCTCTTTAC-3′ (SEQ ID NO: 11) |
| | RV (Reverse) | 5′-CTGACTGGTGGTATCTCTTGACC-3′ (SEQ ID NO: 12) |
| KAI1 (CD82) | FW (Forward) | 5′-CACTACAACTGGACAGAGAACGAG-3′ (SEQ ID NO: 13) |
| | RV (Reverse) | 5′-TGTAGTCTTCAGAATGAATGTACCG-3′ (SEQ ID NO: 14) |

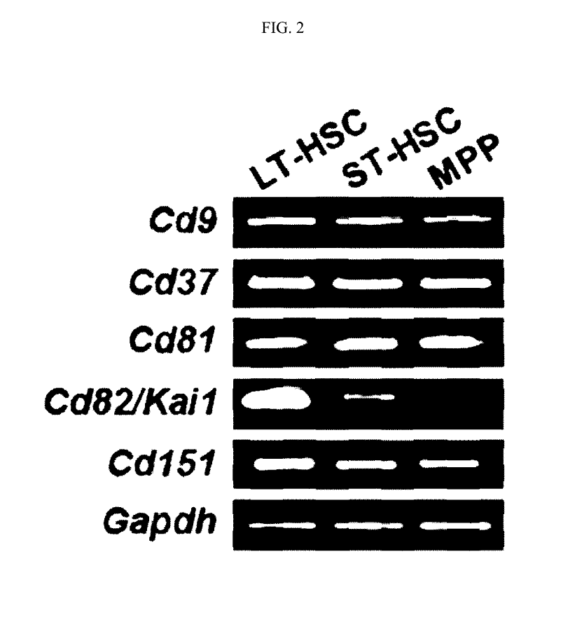

As shown in FIG. 2, it can be seen from the result that most of the tetraspanins were expressed in all the LT-HSCs, ST-HSCs and MPPs, but the KAI1 (CD82) was only specifically expressed in the LT-HSCs.

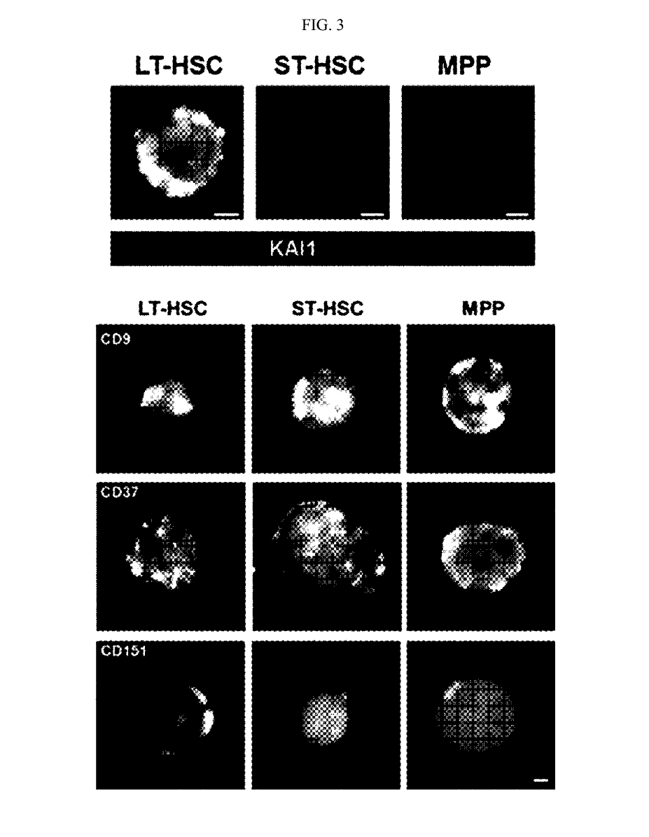

Subsequently, fluorescence staining was performed on the cells obtained in Example 1-1, and therethrough, as shown in FIG. 3, it can be seen that the KAI1 was only expressed in the LT-HSCs while CD9, CD37, and CD151 were expressed in all of the LT-HSCs, ST-HSCs, and MPPs.

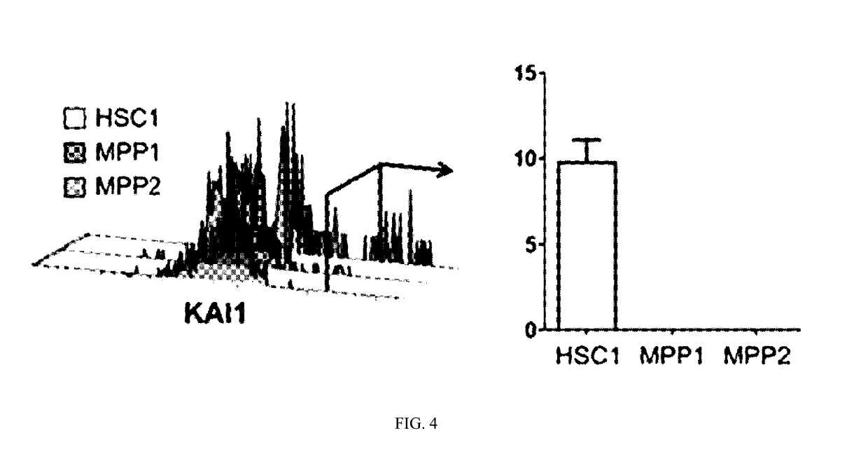

Moreover, in a recently published research paper, using quiescent HSC markers with a higher purity, HSC1 or quiescent HSC was designated as CD48-CD229-CD244-CD150+LSK, MPP1 was designated as CD48-CD229-CD244-CD150-LSK, and MPP2 was designated as CD48-CD229+CD244-CD150-LSK, and as shown in FIG. 4, staining of KAI1 with the same markers used previously shows that approximately 10% of KAI1(+) cells were expressed in HSC1 (quiescent HSC) but no KAI1(+) cells were expressed in MPP1 and MPP2.

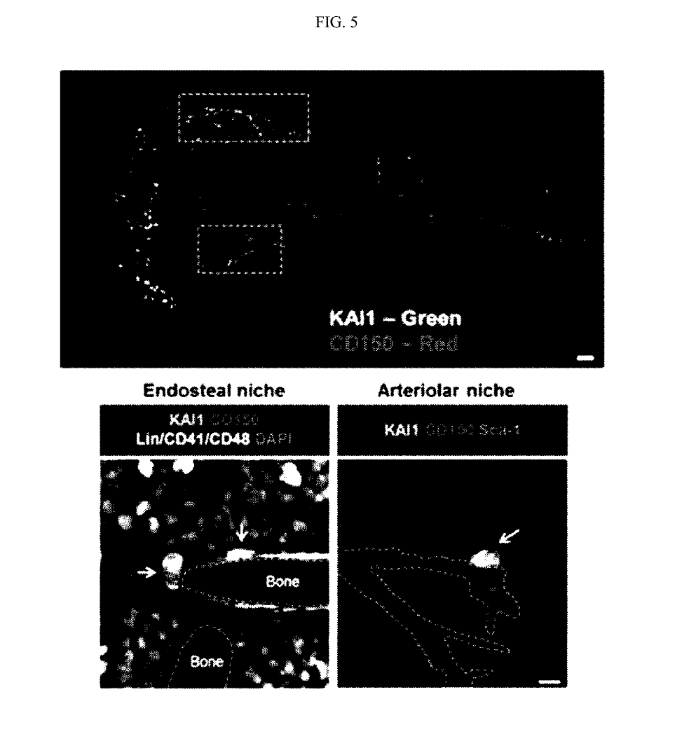

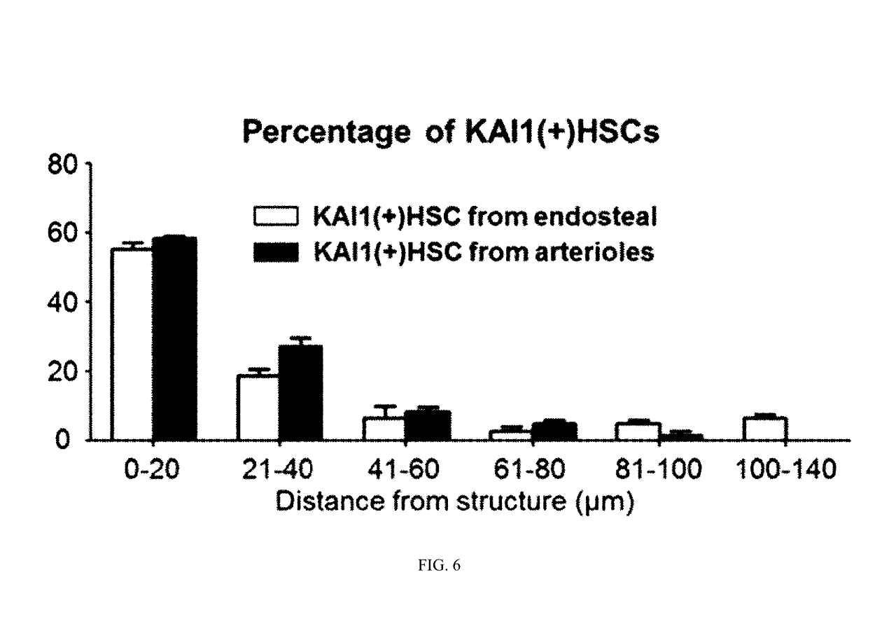

Furthermore, to determine the location of KAI1(+)LT-HSC in a bone, the bone of a C57BL/6 mouse was stained. Bone staining was performed by fixing the femur of the C57BL/6 mouse with 4% paraformaldehyde for 24 hours. Afterwards, the resulting femur of the C57BL/6 mouse was exposed to a decalcification solution at room temperature for 24 hours to prepare a slide using a paraffin block. The paraffin slide was stained through a de-paraffinization process. As a result of examination of cells in which lineage, CD41 and CD48 as LT-HSC markers, and CD150 and KAI1 as negative markers were simultaneously expressed, as shown in FIGS. 5 and 6, it can be seen that most of the cells were located in bone linings. More specifically, most of Lin(−)CD41(−)CD48(−)CD150(+)KAI1(+) cells were located in the endosteal niche and the arteriolar niche. Meanwhile, according to the prior references, the endosteal niche and the arteriolar niche are known as places retaining quiescent HSCs.

### Example 2

### Confirmation of Increase in LT-HSC Cell Count in Bone-Marrow (BM) According to KAI1(CD82) Expression

### 2-1. Preparation of KAI1 Knock-Out Mice

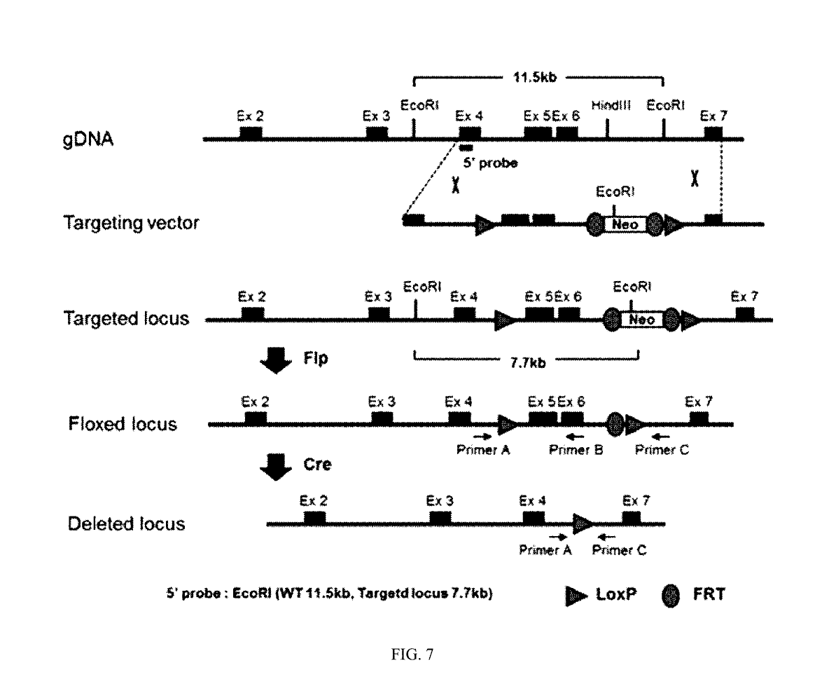

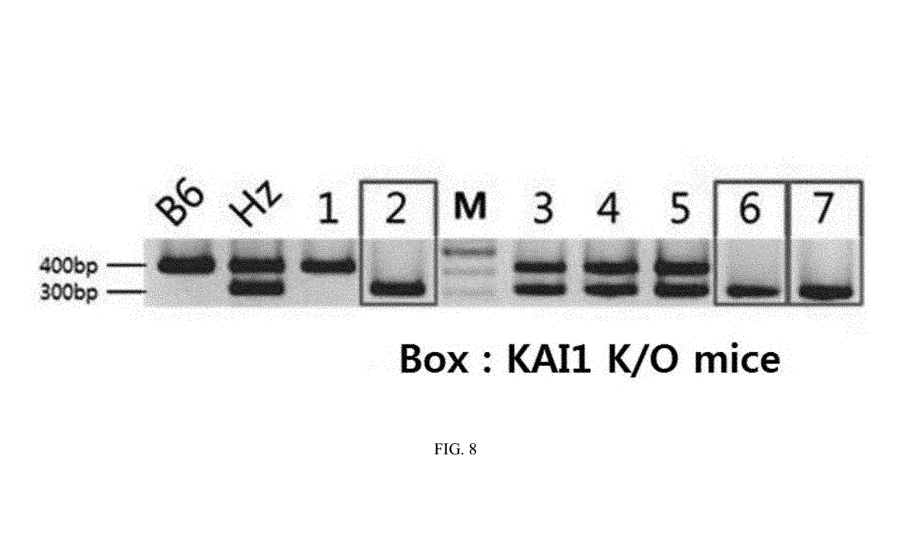

The inventors themselves were the first in the world to prepare KAI1 knock-out mice . More specifically, the KAI1 knock-out mouse was prepared by targeting the 5^{th} and 6^{th} genes of KAI1, and then identified using mouse genomic DNA and primers listed in Table 2 below (refer to FIGS. 7 and 8). In the drawings, 400 bp represents WT, and 300 bp represents the KAI1 K/O mouse.

TABLE 2

| Type | Primer sequence |
|---|---|
| Primer A | 5′-GGGTCCCCTAGGAAATTCAA-3′ |
| | (SEQ ID NO: 15) |
| Primer B | 5′-ATGATGCAGATGTTCTCTCAGGGTG-3′ |
| | (SEQ ID NO: 16) |
| Primer C | 5′-ACAGGGGACTCACCC TACAAGG-3′ |
| | (SEQ ID NO: 17) |

2-2. Comparison of Cell Counts in BM between WT and KAI1 K/O Mice

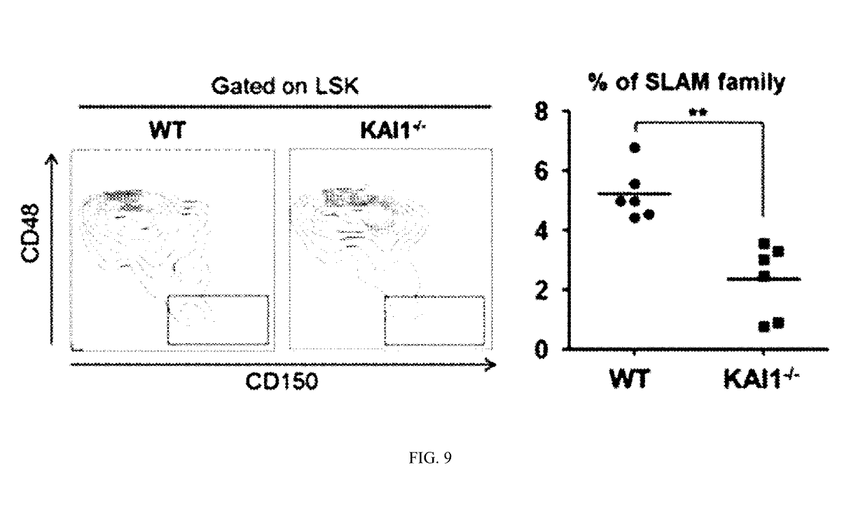

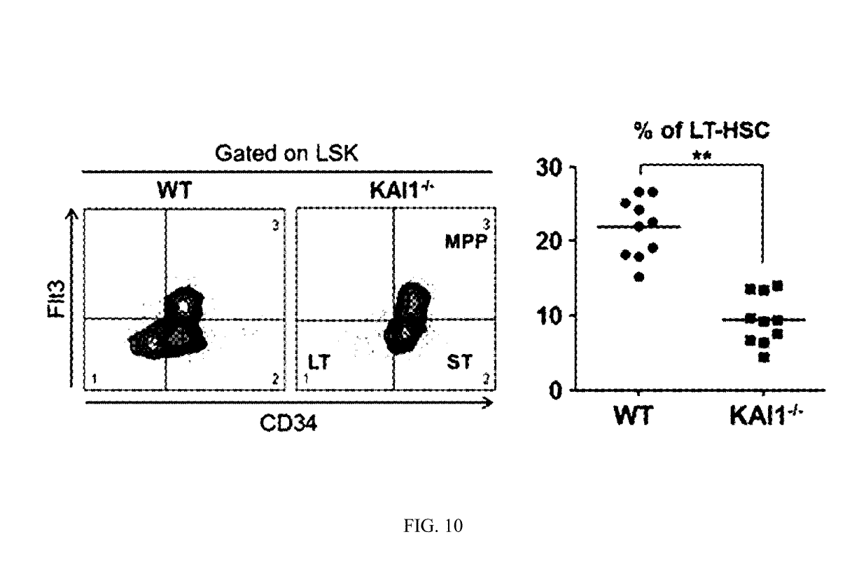

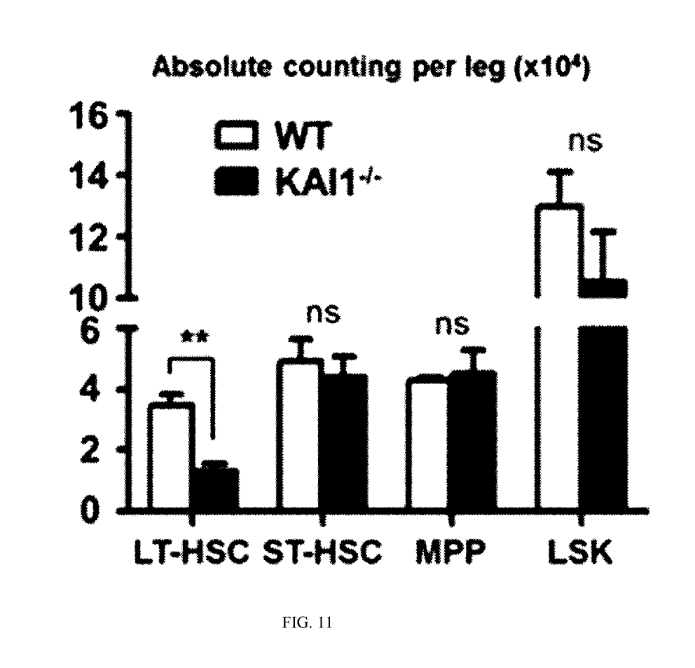

First, LT-HSC, ST-HSC, and MPP cell counts in the BM between the WT and the KAI1 K/O mice were compared using FACS analysis, showing that, as shown in FIG. 9, a cell count of CD48(−)CD150(+)LSK, which is a LT-HSC marker, was lower in the KAI1 K/O mice than in the WT. In addition, as shown in FIG. 10, a cell count of CD34(−)Flt(−)LSK, also known as an LT-HSC marker, was lower in the KAI1 K/O mice than the WT, and as shown in FIG. 11, the absolute cell counts were lower in the KAI1 K/O mice.

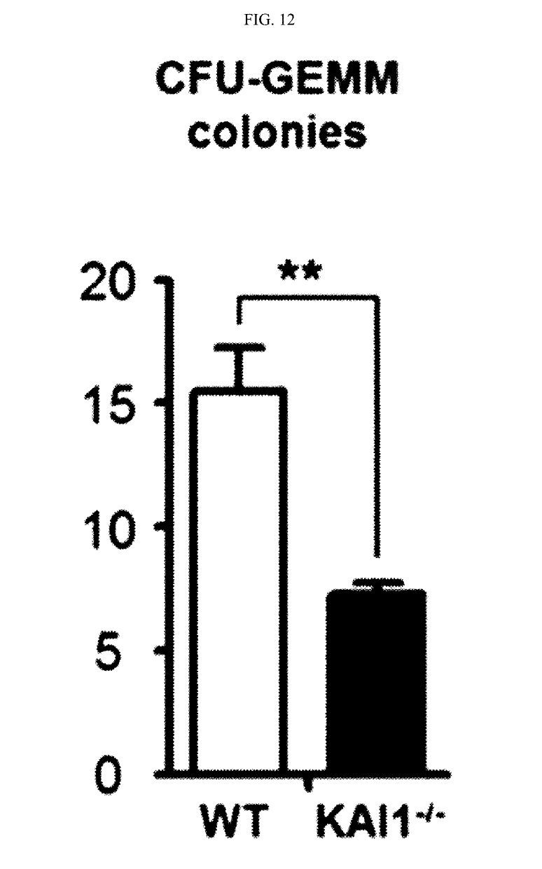

Subsequently, a colony forming unit (CFU) assay was carried out on BM cells of WT and KAI1 K/O mice using MethoCult™ GF M343 (Stem Cell Technologies, Cat No. 03434), and therethrough, as shown in FIG. 12, it can be confirmed that multi-potential granulocytes, erythroids, macrophages, and megakaryocyte progenitors (CFU-GEMM) were reduced in the KAI1 K/O mice than WT.

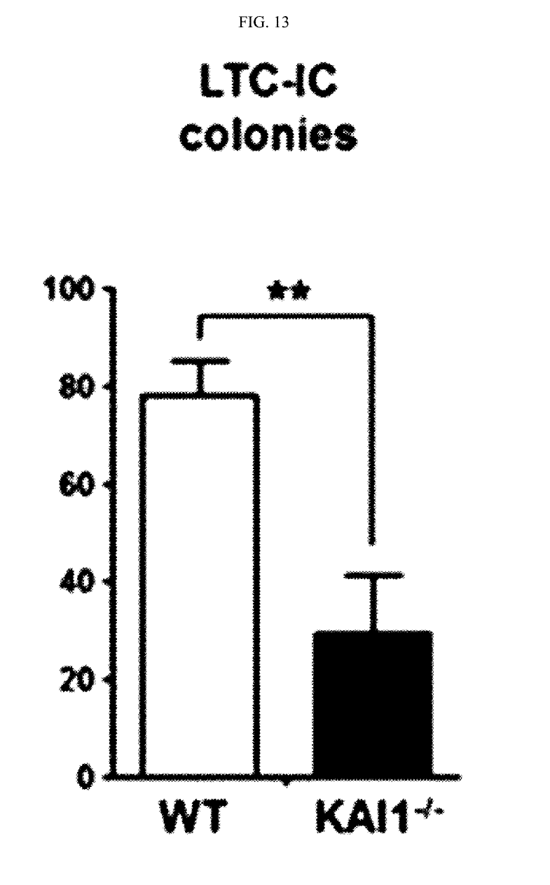

Afterwards, to quantify LT-HSCs, a long term culture-initiating cell (LTC-IC) assay was carried out. More specifically, mouse stromal cells (OP9) were exposed to irradiation at 30 Gy and used as feeder cells. In addition, LSK cells were sorted from BM in each of WT and the KAI1 K/O mice, and each cell was cultured on the feeder cell. The culturing was carried out in a hydrocortisone-containing MyeloCult medium (StemCell technology), and half-media changes were carried out once a week. After a total of four weeks of culturing, a CFU assay was carried out, and 10 days later, colony counting was performed. Therethrough, as shown in FIG. 13, it can be confirmed that a colony count was lower in the KAI1 K/O mice than in WT. Consequently, it can be seen that the LT-HSC cell count was decreased in the KAI1 K/O mice.

### Example 3

### Confirmation of Cell Cycle of LT-HSCs According to KAI1(CD82) Expression

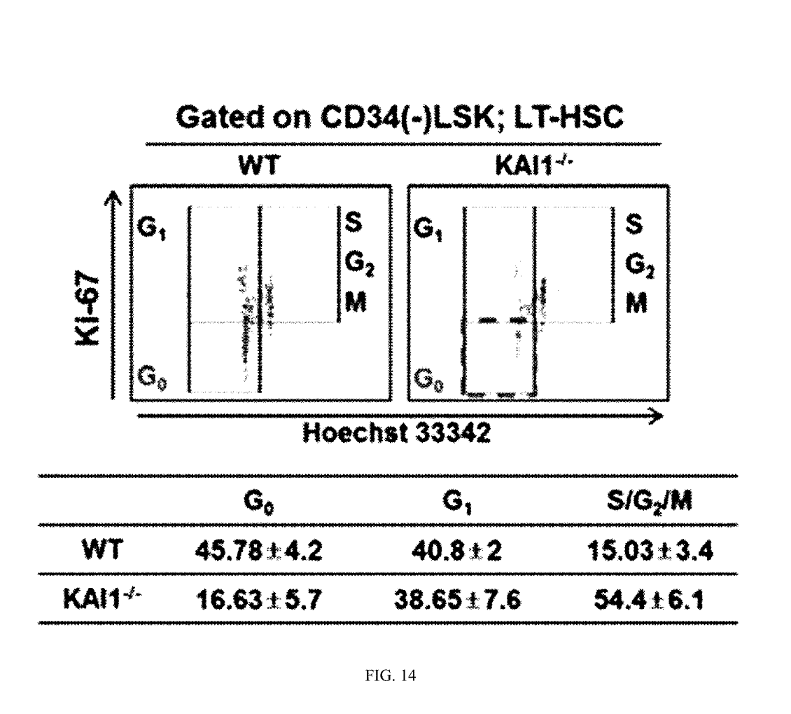

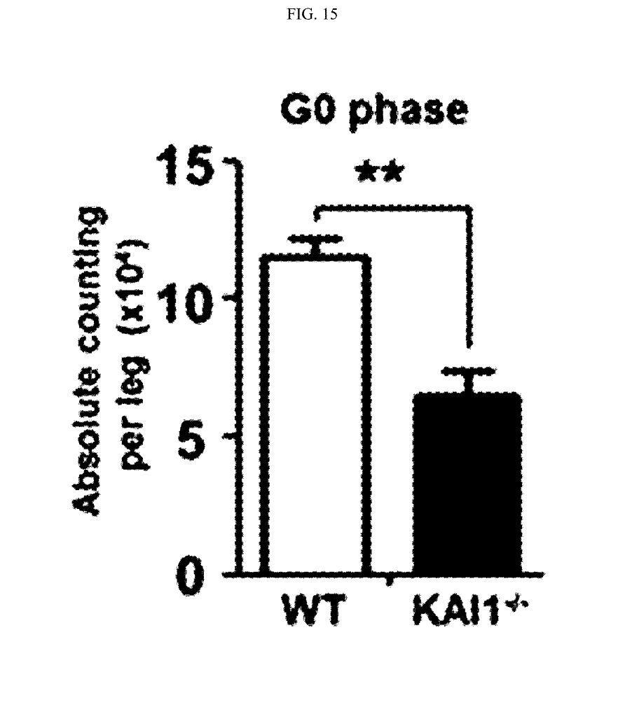

First, bone marrow (BM) of WT and KAI1 K/O mice were obtained and subjected to ki67 and Hoechst 33342 analyses of LT-HSCs (CD34-LSK) using LSR II. Consequently, as shown in FIGS. 14 and 15, it was confirmed that LT-HSCs in G0 phase were less numerous but LT-HSCs in G1/S/G2 phases were more numerous in the KAI1 K/O mice than in WT.

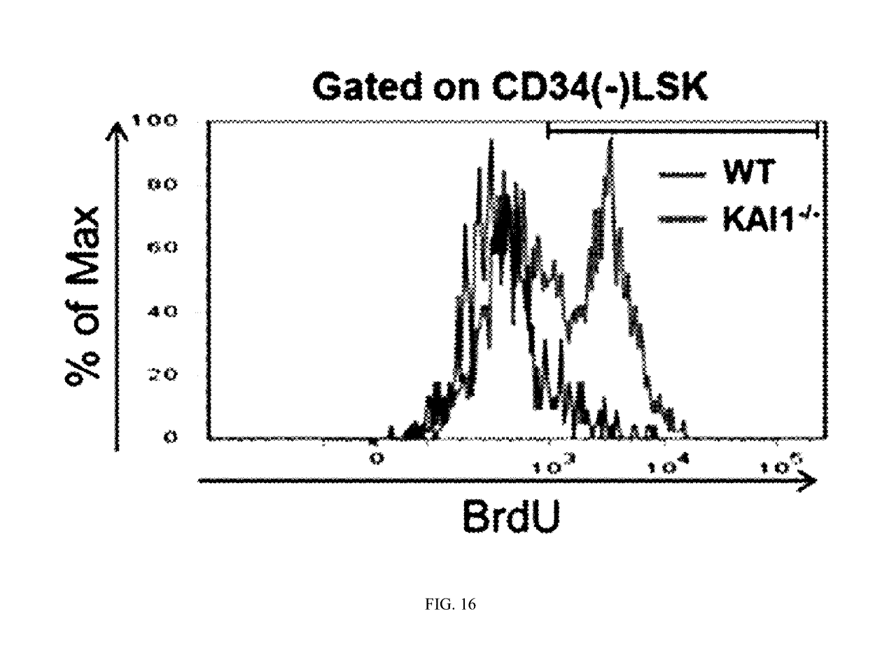

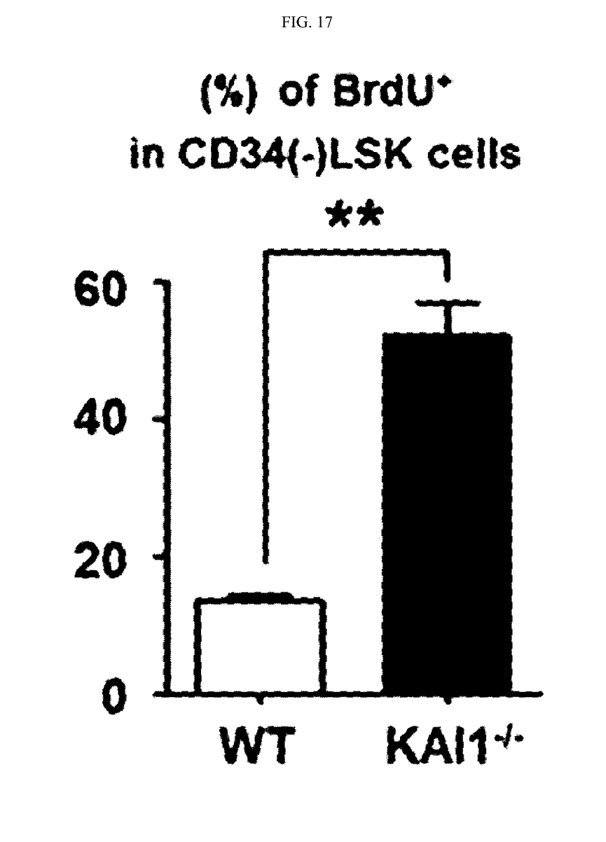

Subsequently, to determine in vivo proliferative activity in WT and KAI1 K/O mice, exogenous BrdU incorporation into genomic DNA was examined. That is, following BrdU treatment, BM cells were obtained from each mouse to detect BrdU positive LT-HSCs, which were considerably more numerous in the KAI1 K/O mice than in WT, as shown in FIGS. 16 and 17.

Afterwards, to detect CDK inhibitors (p21, p27 and p57), which are significant genes for maintaining of HSCs, LT-HSCs of WT and the KAI1 K/O mice were sorted and then had RNA isolated therefrom for RT-PCR. Primer information used herein is listed in Table 3 below.

TABLE 3

| Type | Primer sequence | |
|---|---|---|
| P21 | FW (Forward) | 5′-GAGAACGGTGGAACTTTGACTTC-3′ (SEQ ID NO: 18) |
| | RV (Reverse) | 5′-GTGATAGAAATCTGTCAGGCTGGT-3′ (SEQ ID NO: 19) |
| P27 | FW (Forward) | 5′-CATGAAGAACTAACCCGGGACT-3′ (SEQ ID NO: 20) |
| | RV (Reverse) | 5′-CAGAGTTTGCCTGAGACCCAAT-3′ (SEQ ID NO: 21) |
| P57 | FW (Forward) | 5′-AGATCTGACCTCAGACCCAATTC-3′ (SEQ ID NO: 22) |
| | RV (Reverse) | 5′-GCTCTTGATTCTCGTCCTGCTC-3′ (SEQ ID NO: 23) |

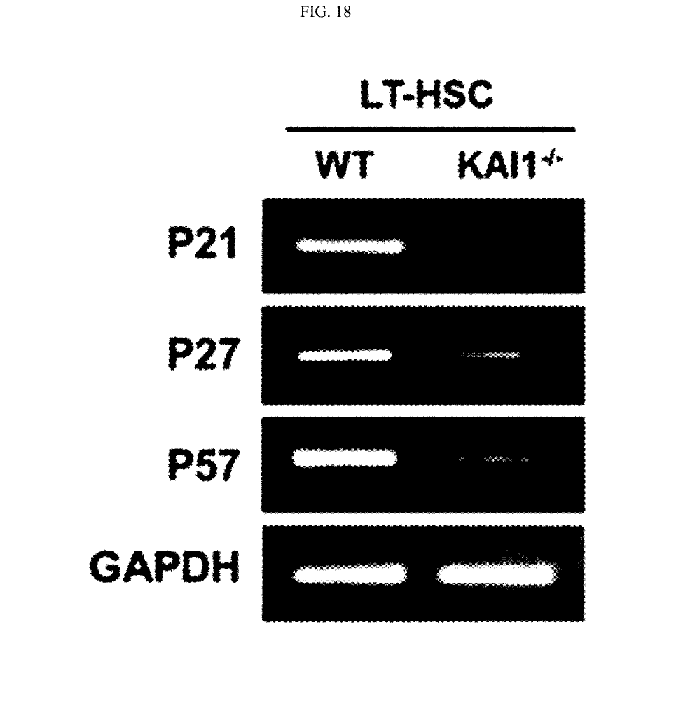

As a result, it can be seen that CDK inhibitors p21, p27, and p57 of LT-HSCs of KAI1 K/O mice were reduced compared to those of WT, as shown in FIG. 18.

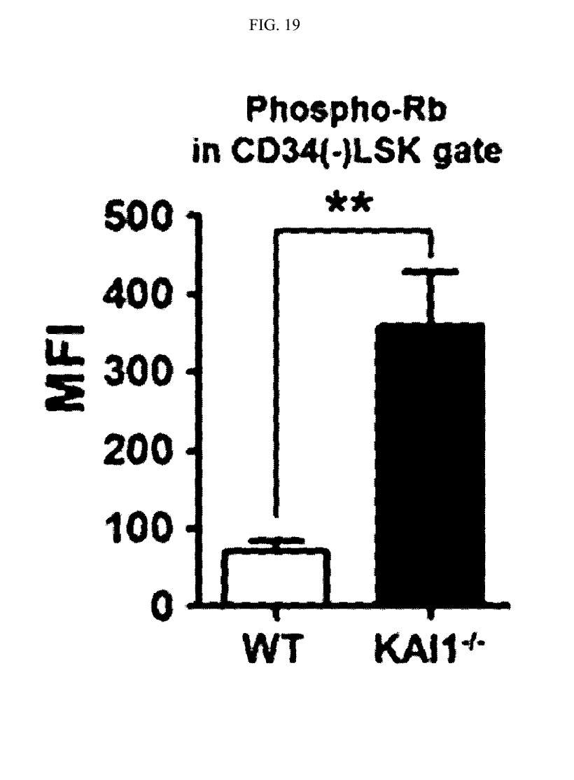

Generally, CDK inhibitors are known to stop the cell cycle by reducing Retinoblastoma (Rb) phosphorylation. Therefore, as a result of confirmation of the Rb phosphorylation of LT-HSCs in WT and the KAI1 K/O mice using FACS analysis, it can be confirmed that Rb phosphorylation was higher in the KAI1 K/O mice than in WT, as shown in FIG. 19.

From the above results, it can be seen that quiescence was reduced in the KAI1 K/O mouse LT-HSCs.

### Example 4

### Identification of Signaling Mechanism for KAI1(CD82) to Maintain Quiescence of LT-HSCs

### 4-1. Preparation of Cell Line

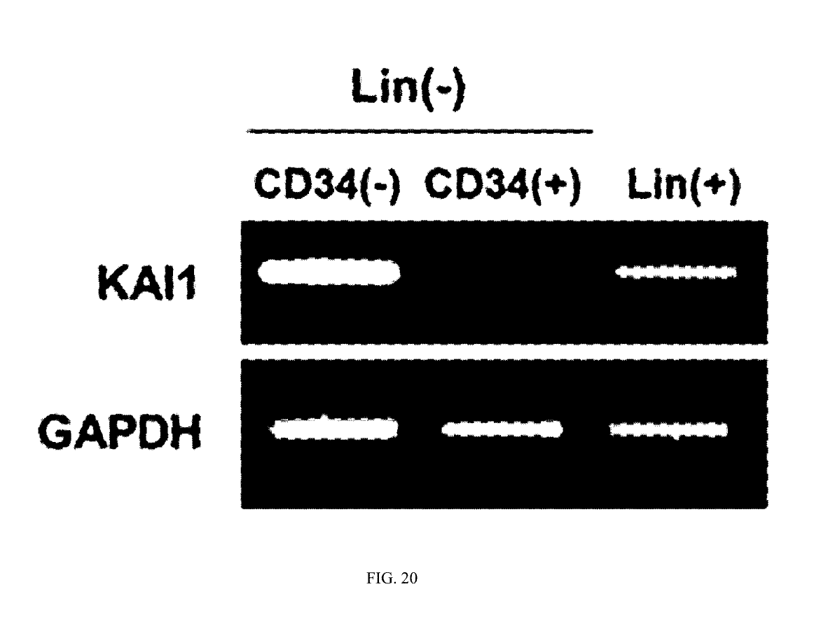

To study a mechanism of KAI1 for maintaining quiescence of LT-HSCs, an EML cell line which has been widely used in research on hematopoietic stem and progenitor cells (HSPCs) was used. The EML cell line was quiescent Lin(−)CD34(−) cells, and Lin(−)CD34(+) cells are cells exhibiting high proliferative activity, in which the cell cycle stays active. The two populations were sorted to compare KAI1 gene expression using the KAI1 primers listed in Table 1, and as a result, it can be confirmed that KAI1 was highly expressed in quiescent Lin(−)CD34(−) cells but not expressed in Lin(−)CD34(+) cells, as shown in FIG. 20.

### 4-2. KAI1 Knock-Down or Overexpression in EML Cells

Knock-down or overexpression of KAI1 was carried out in EML cells using a lentivirus.

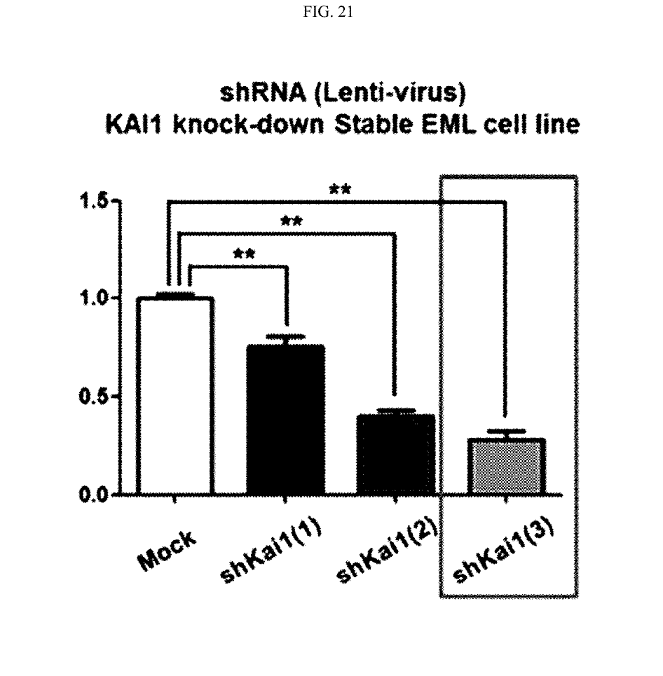

First, shRNA constructs were used for KAI1 knock-down (Sigma-Aldrich, TRCN0000042409, TRCN0000042410, and TRCN0000042411). After transduction, selection was carried out with puromycin (2 μg/ml). In addition, the KAI1 knock-down was confirmed using real-time PCR (qPCR), and information on KAI1 primers only for qPCR used herein is listed in Table 4 below. As a result, it can be confirmed that KAI1 was knocked down in EML cells by treatment with shRNA constructs, as shown in FIG. 21.

TABLE 4

| Type | Primer sequence | |
|---|---|---|
| KAI1 | FW (Forward) | 5′-GCCTGGGACTACGTGCAG-3′ |
| | | (SEQ ID NO: 24) |
| | RV (Reverse) | 5′-CCTCGTTCTCTGTCCAGTTGT-3′ |
| | | (SEQ ID NO: 25) |

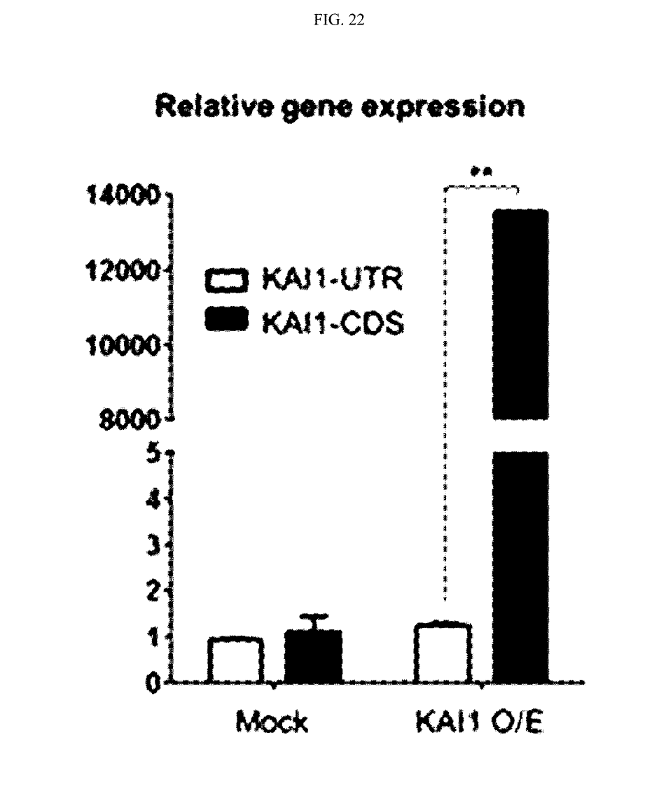

Afterwards, for KAI1 overexpression (O/E), KAI1 cDNA was inserted into pLenti 6.3/v5-Dest (Invitrogen) and then cloning was performed. 293FT cells were transfected with the KAI1 pLenti 6.3/V5-Dest using a ViraPower packaging mix (Invitrogen) and polyethylenimine (PEI). In addition, after 48 hours, viral supernatants were acquired and passed through a filter to concentrate viruses using ultracentrifugation. The viruses obtained thereby were transduced to the EML cells, and after 48 hours, selection was carried out using blasticidin S (5 μg/ml). As a result of examining KAI1 increase using the primers listed in Table 4, it can be confirmed that KAI1 was overexpressed, as shown in FIG. 22.

### 4-3. Comparative Experiment Between KAI1 Knock-Down and KAI1 Overexpressed EML Cells

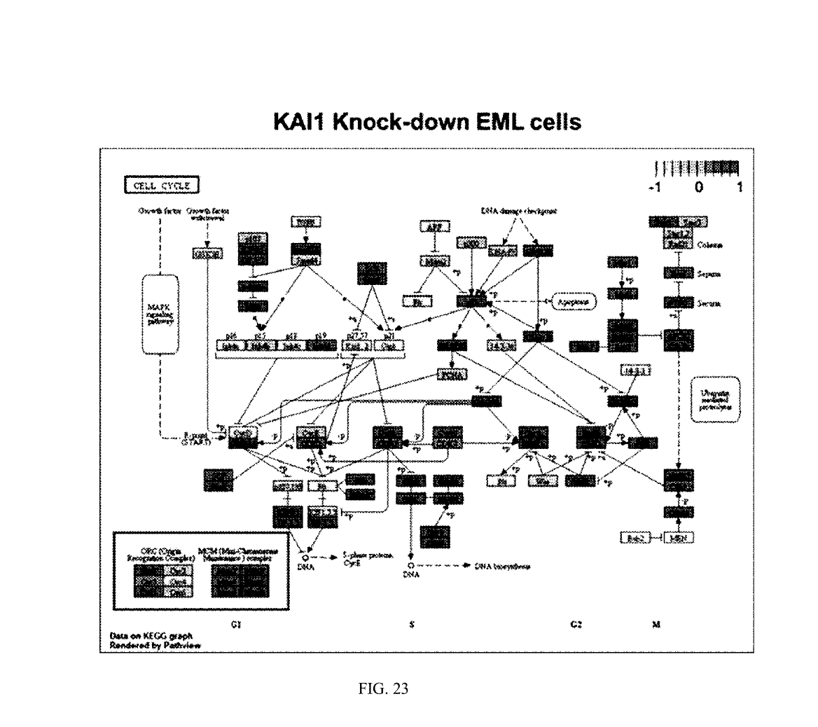

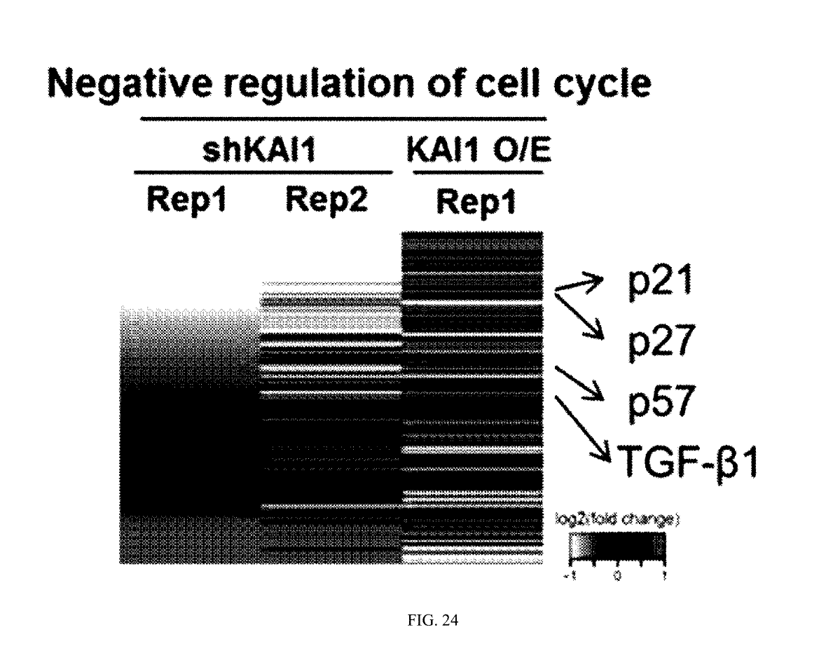

First, the KAI1 knock-down and KAI1 overexpressed EML cells obtained in Example 4-2 were subjected to RNA sequencing analysis. As a result, it can be confirmed that a CDK inhibitor was decreased and upstream TGF-β thereof was decreased in KAI1 knock-down EML cells, as shown in FIG. 23. In addition, it can be confirmed that the number of genes in the negative cell cycle were reduced in the KAI1 knock-down EML cells, whereas the number of genes in the negative cell cycle were increased in KAI1 overexpression EML cells, as shown in FIG. 24.

Subsequently, the increase/decrease in TGF-β1 and TGFR2 was confirmed using RT-PCR and Western blotting. Primer information used herein is shown in Table 5 below.

TABLE 5

| Type | Primer sequence | |
|---|---|---|
| TGF-01 | FW | 5′-CGGACTACTATGCTAAAGAGGTCAC-3′ |
| | (Forward) | (SEQ ID NO: 26) |
| | RV | 5′-GAGTTTGTTATCTTTGCTGTCACAAG-3′ |
| | (Reverse) | (SEQ ID NO: 27) |
| TGFOR2 | FW | 5′-CATCTTCTACTGCTACCGTGTCC-3′ |
| | (Forward) | (SEQ ID NO: 28) |
| | RV | 5′-ATGTTCTCATGCTTCAGGTTGAT-3′ |
| | (Reverse) | (SEQ ID NO: 29) |

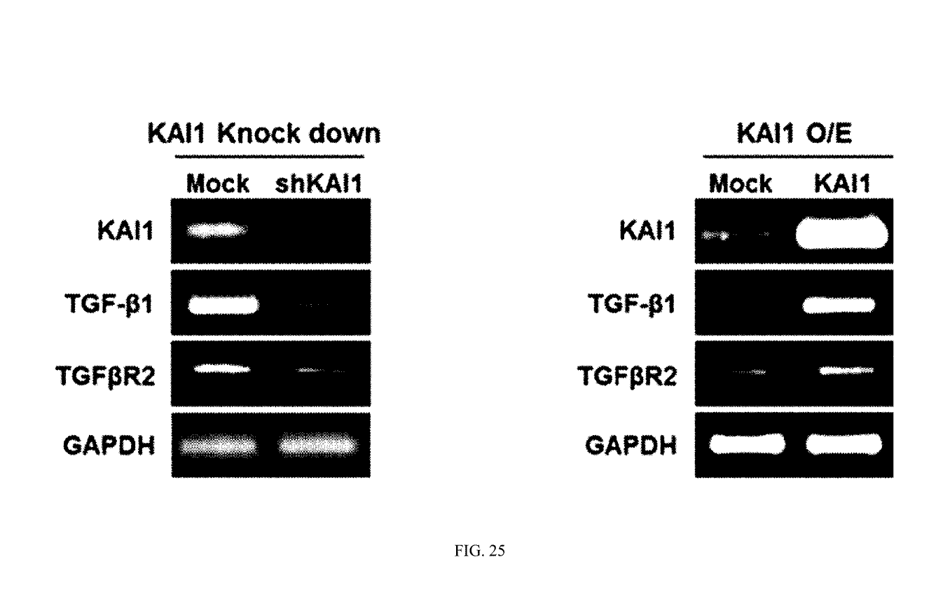

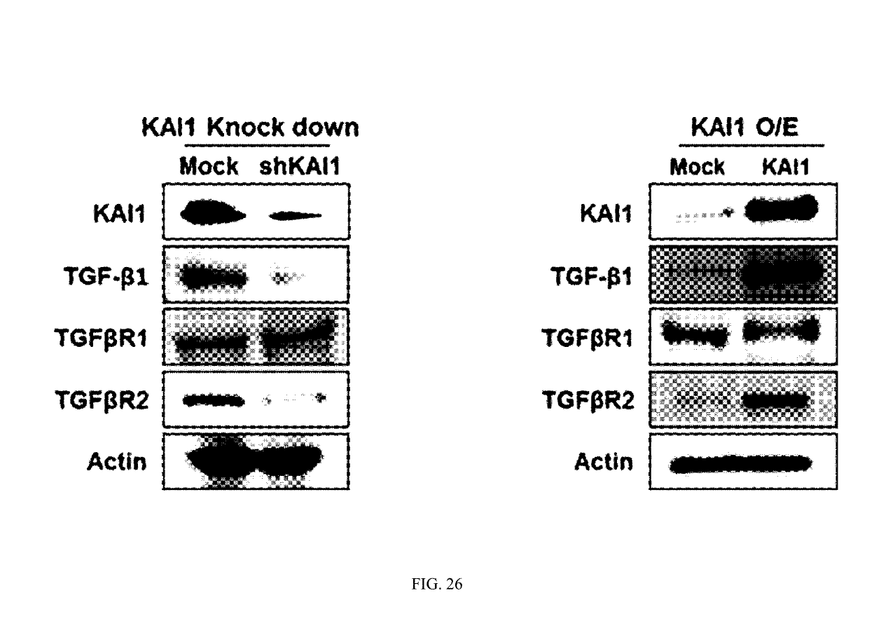

As shown in FIGS. 25 and 26, it can be confirmed that TGF-β1 and TGFβR2 expression was reduced in KAI1 knock-down EML cells, whereas TGF-β1 and TGFβR2 expression was increased in KAI1 Over-expressed EML cells.

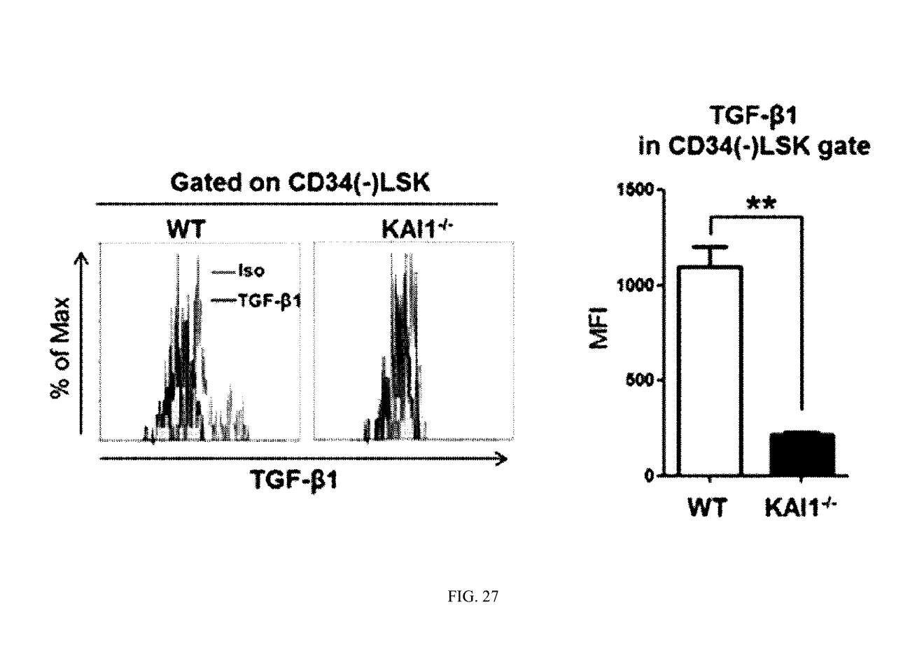

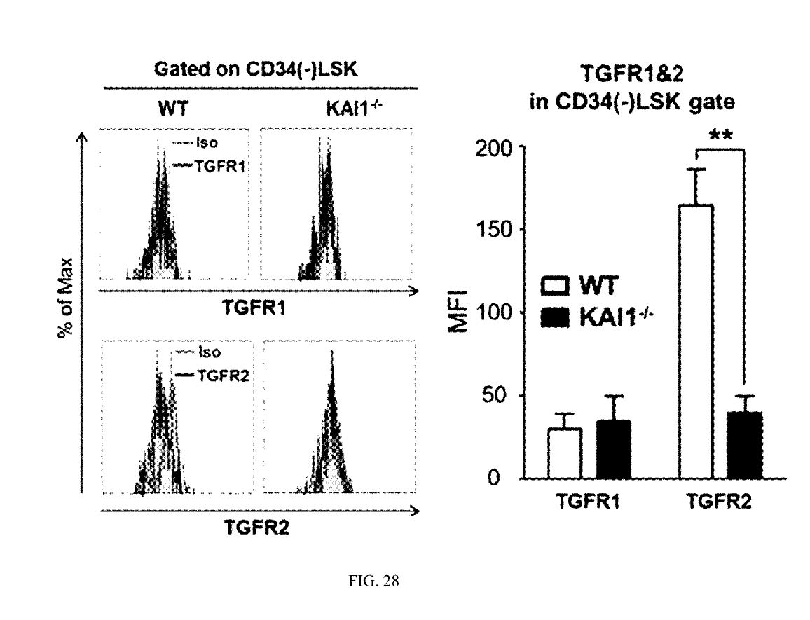

Subsequently, BM cells were obtained from actual femurs and tibias of WT and KAI1 K/O mice, and therefrom peripheral blood mononuclear cells (PBMCs) were isolated using histopaque-1083 (Sigma-Aldrich) for FACS analysis. As a result of comparing TGF-β1 and TGFR2 positive cells in LT-HSCs, which are Flt3-CD34-LSK cells, it can be confirmed that the TGF-β1 and TGFR2 positive cells were reduced in the KAI1 K/O mice, as shown in FIGS. 27 and 28.

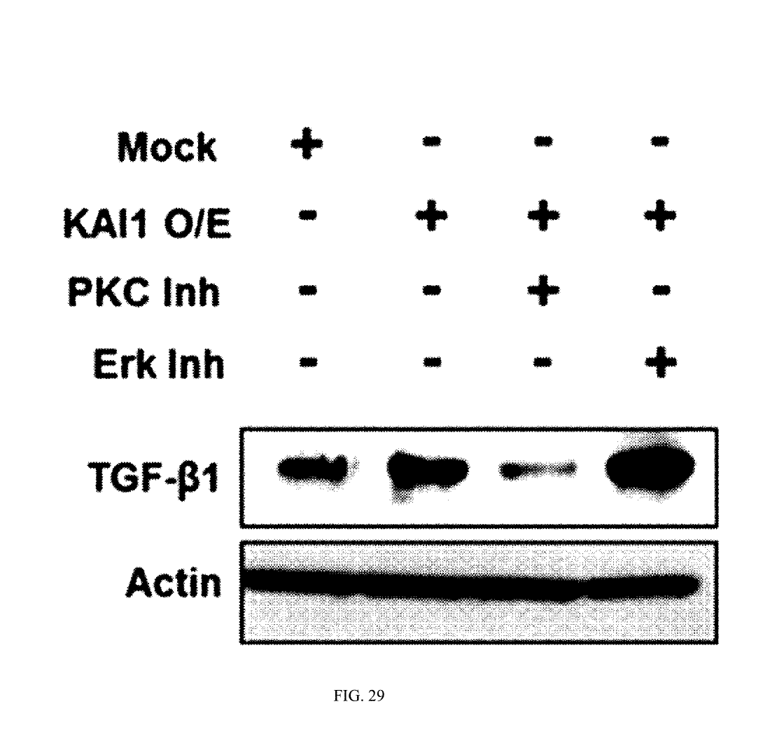

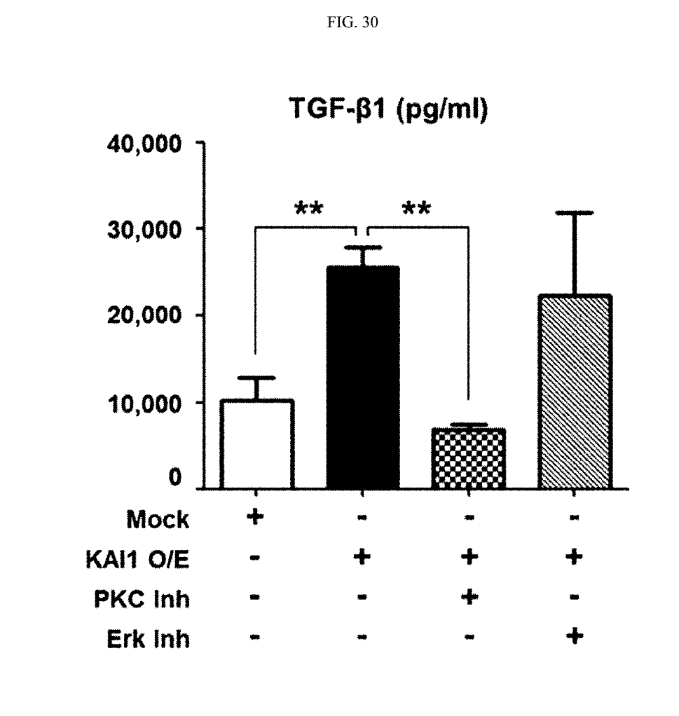

Afterwards, since TGF-β1 has been conventionally known to be increased by PKC and Erk pathways, KAI1 overexpressed EML cells were treated with their inhibitors to find a link between increasing TGF-β1 expression and KAI1, and TGF-β1 expression and secretion were confirmed using Western blotting. Consequently, as shown in FIGS. 29 and 30, it can be confirmed that KAI1 increased the TGF-β1 expression and secretion via PKC, not Erk.

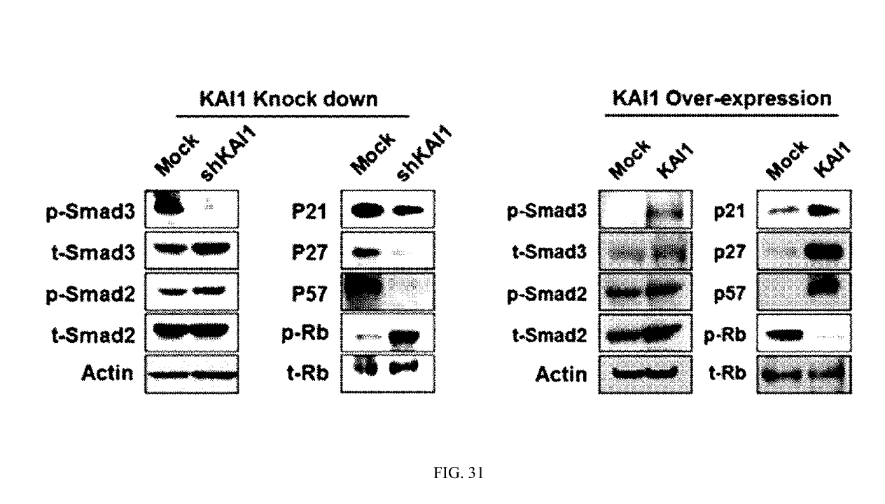

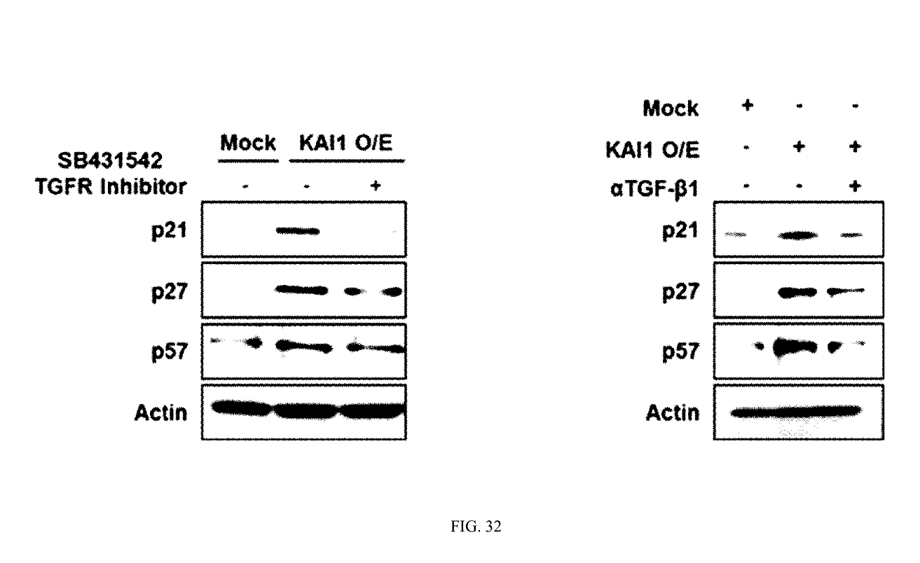

Then, in KAI1 knock-down and KAI1 overexpressed EML cells, Smad2 and Smand3 phosphorylation as downstream pathways of TGF-β1/TGFR and the increase/decrease in expression of CDK inhibitors such as p21, p27, and p57 were compared using Western blotting. As a result, as shown in FIG. 31, it can be confirmed that the p21, p27 and p57 as well as Smad3 phosphorylation were reduced in the KAI1 knock-down cells. On the other hand, the Rb phosphorylation was increased. However, it can be confirmed that the opposite results were attained in the overexpression of KAI1.

In addition, it can be determined that the p21, p27 and p57 increased by the KAI1 overexpression EML cells were decreased again in the TGFR inhibitor-treated group by treatment of the KAI1 overexpression EML cells with a TGFR inhibitor, confirming whether the CDK inhibitors were increased by TGF-β1. In addition, it can be confirmed that the result was obtained using even a neutralizing antibody of TGF-β1.

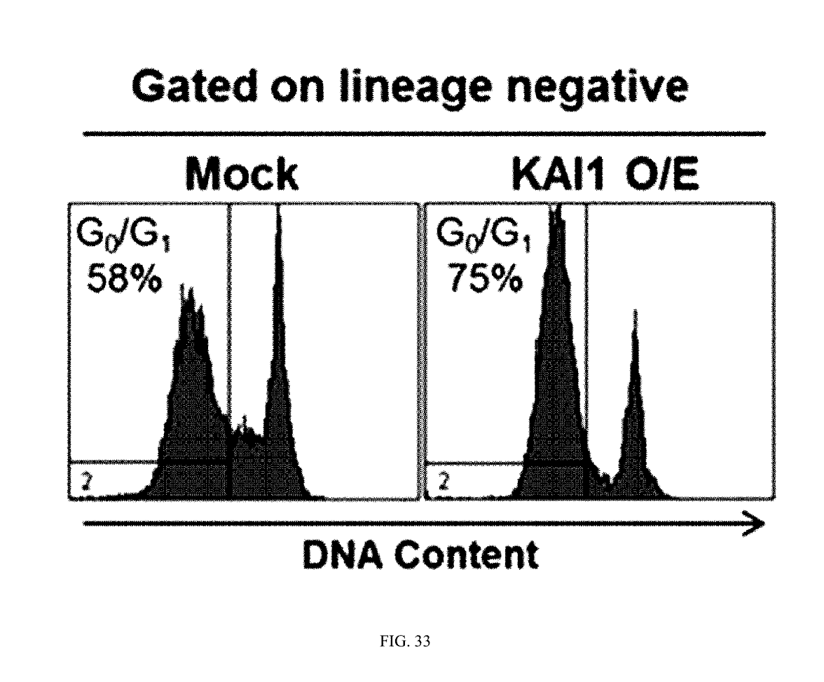

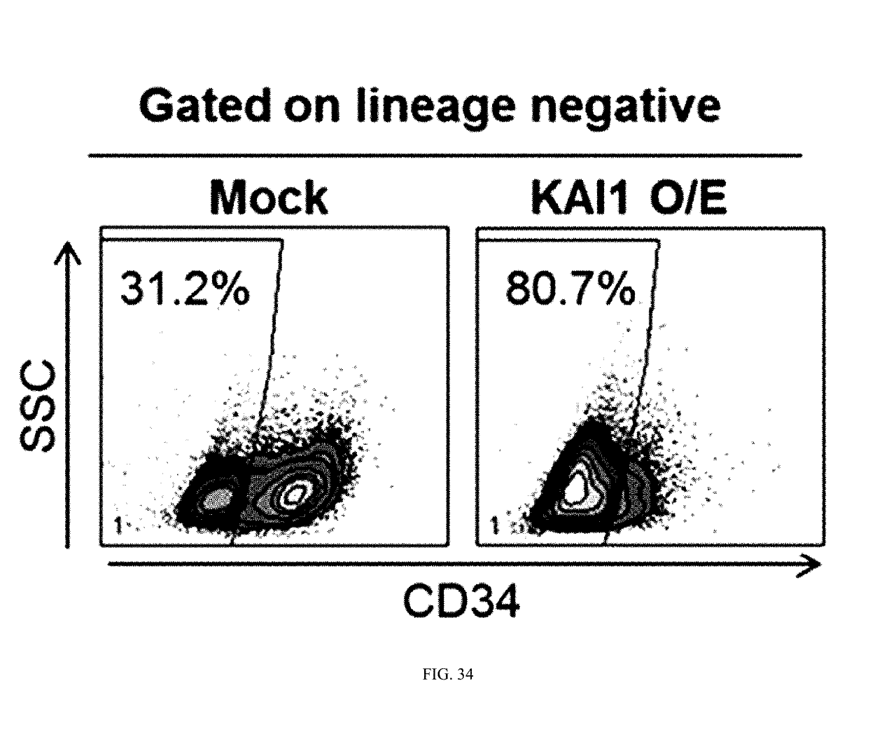

Moreover, to confirm whether the change in CDK inhibitor by the KAI1 overexpression actually affected a cell cycle, FACS was carried out on Mock/, KAI1 O/E EML to compare cell cycles. As a result, as shown in FIGS. 33 and 34, it can be confirmed that number of G0/G1 phase positive cells was increased (58% vs 75%) in KAI1 O/E EML, and a Lin− a Lin-CD34− population was increased more than CD34+ population known to activate cell cycling in EML cells.

### Example 5

### Confirmation of Role of KAI1(CD82) in LT-HSCs When BM was Reconstructed After Ablation

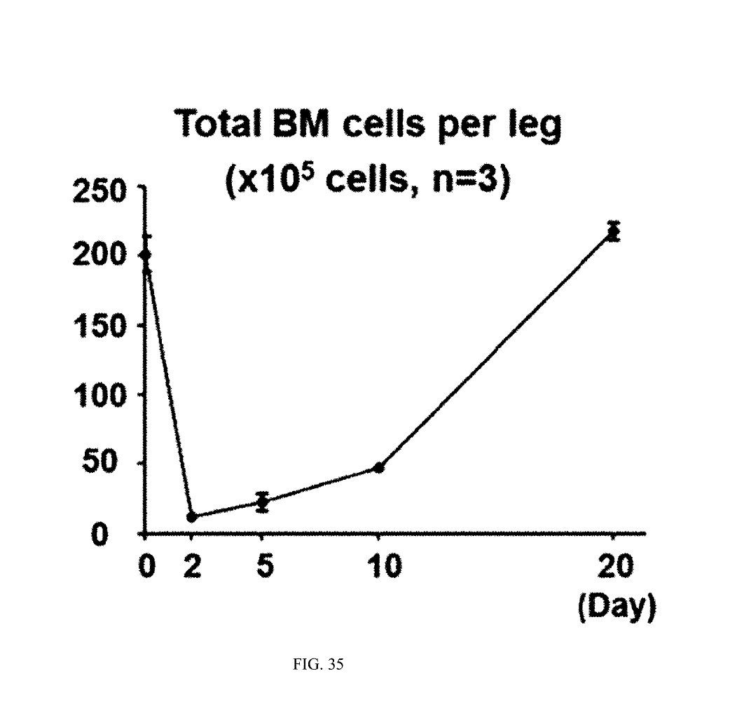

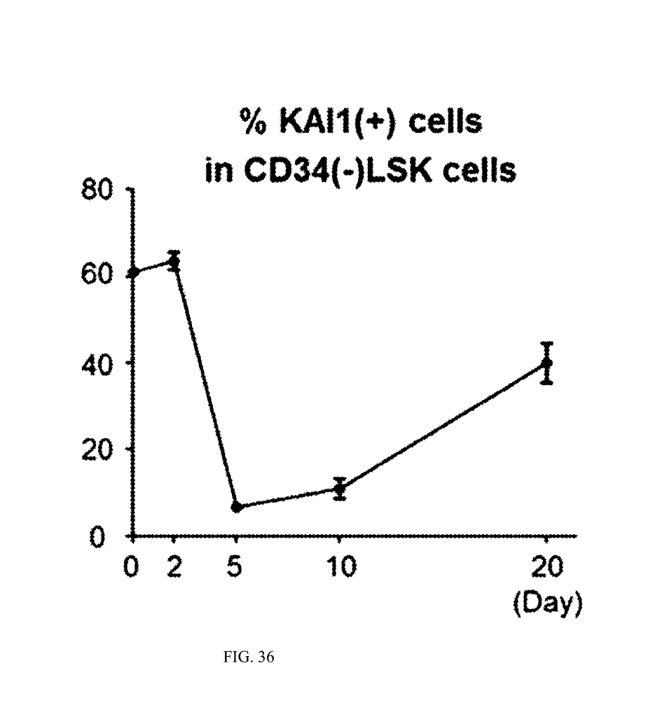

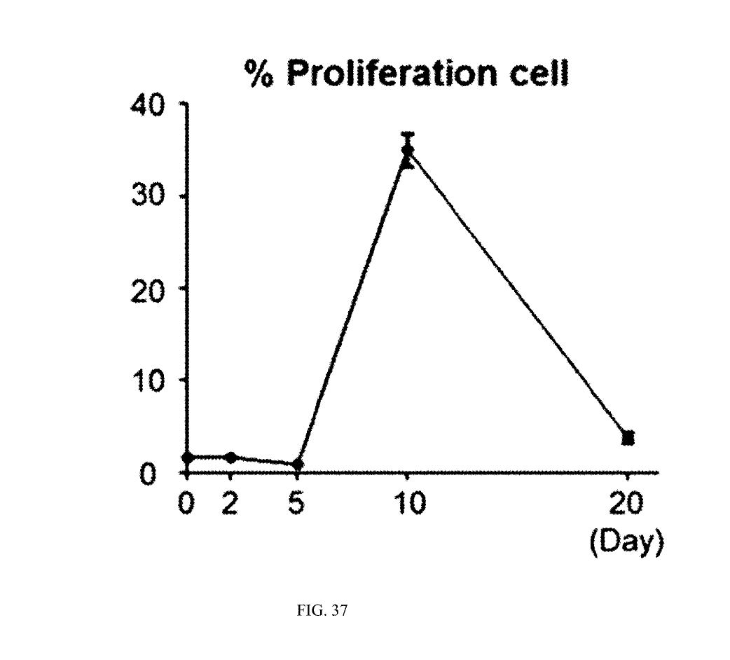

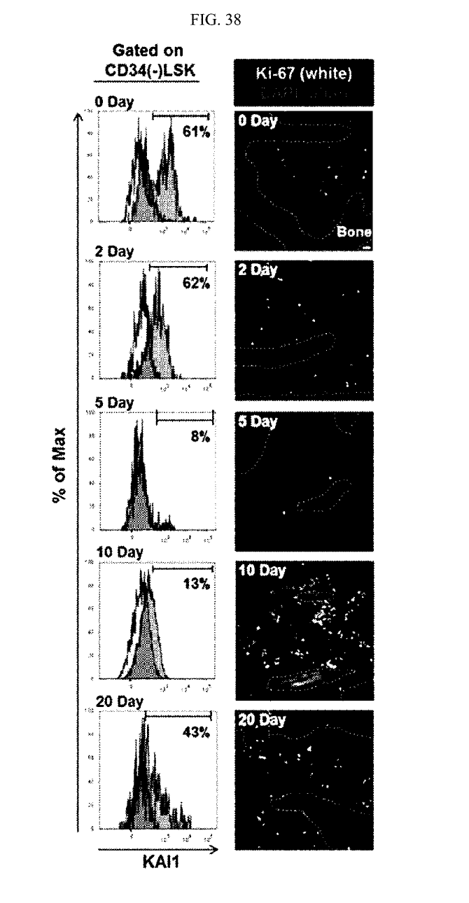

To confirm the role of KAI1 in BM reconstruction and maintenance of quiescent LT-HSCs after ablative intervention, 5-FU was injected into WT mice exposed to sub-lethal irradiation, and then continuous changes in KAI1-positive LT HSCs and proliferating blood cells in BM were confirmed. As a result, as shown in FIG. 35, it can be confirmed that since 5-FU induces apoptosis of actively cycling progenitors or blood cells, the number of cells in the BM was rapidly decreased. In addition, as shown in FIGS. 36 to 38, it can be confirmed that to provide new cells in response to the decrease in cells in BM due to the 5-FU treatment, KAI1 expression in LT-HSCs started to decrease on the second day, went to the lowest level on the 5^{th} day, and then recovered until the second week. Moreover, it can be confirmed that the number of cells starting to differentiate from BM was increased starting from the 5^{th} day due to the decrease in LT-HSC KAI1 expression.

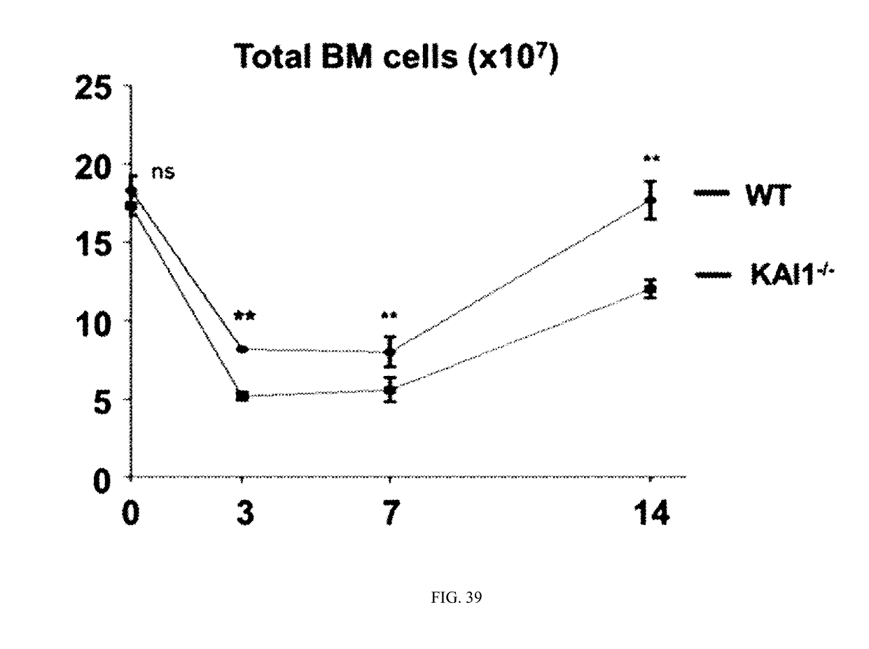

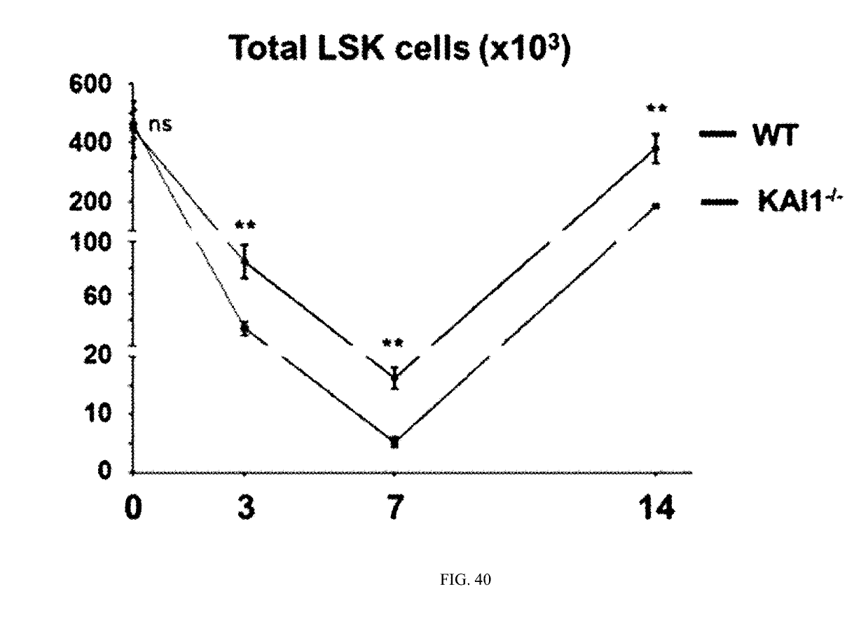

Furthermore, to confirm that KAI1 is important in LT-HSCs for recovering hematopoietic stem cells after the ablation of BM cells, total BM and LSK cell recovery in KAI1^{-/-} and WT mice after sub-lethal irradiation were compared. As a result, as shown in FIGS. 39 and 40, it can be confirmed that the recovery of total BM and LSK cells was lower in the KAI1 mice than in WT.

Therethrough, it can be seen that the decrease in KAI1 in LT-HSCs was significant in BM reconstruction after ablation.

### Example 6

Confirmation of Role of KAI1(CD82) in LT-HSCs and Lone Term Reconstitution after BM Cell Transplantation

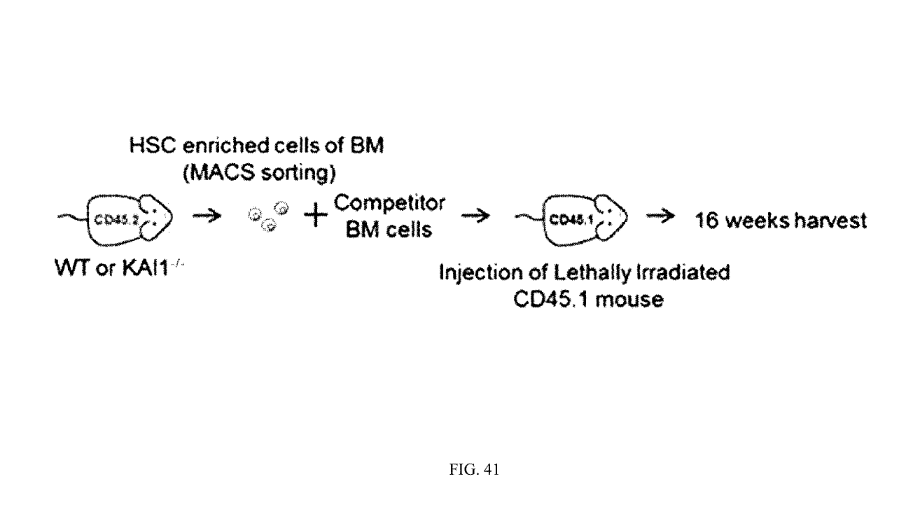

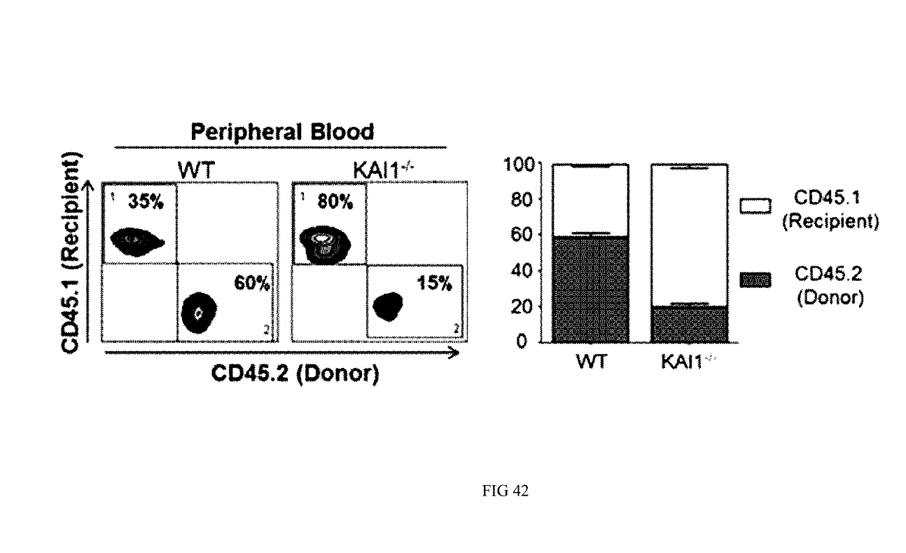

First, to determine if KAI1 affects long term repopulating capacity of HSCs, a competitive BM transplantation (BMT) experiment was carried out, and the process of the experiment is schematically shown in FIG. 41. That is, HSPC-enriched 1×10⁵ Lin(−) BM cells (CD45.2, WT)+5×10⁵ competitor cells (CD45.1) or HSPC-enriched 1×10⁵ Lin(-) BM cells (CD45.2, KAI1^{-/-} mice)+5×10⁵ competitor cells (CD45.1) were transplanted into lethally irradiated recipient mice (CD45.1), and after 16 weeks, BM cells were harvested for FACS analysis. Consequently, as shown in FIG. 42, it can be confirmed that KAI1^{-/-} cells exhibited a lower repopulating capability than WT cells.

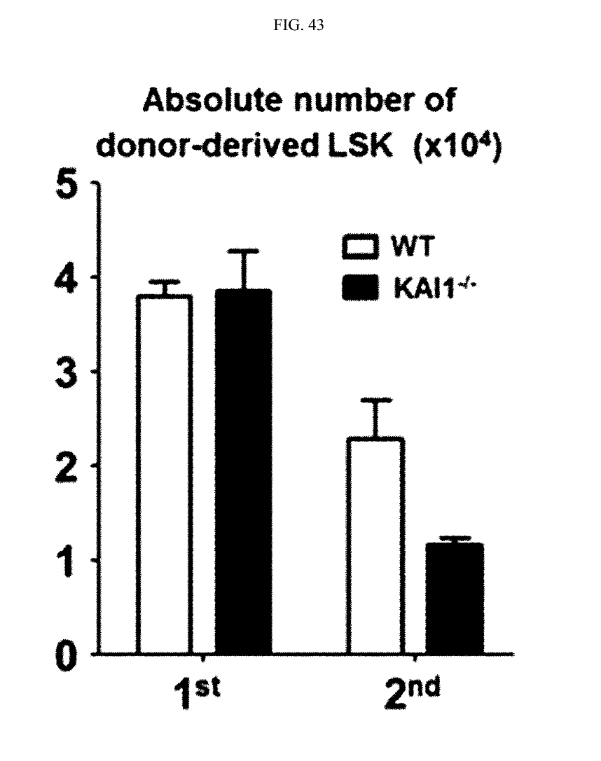

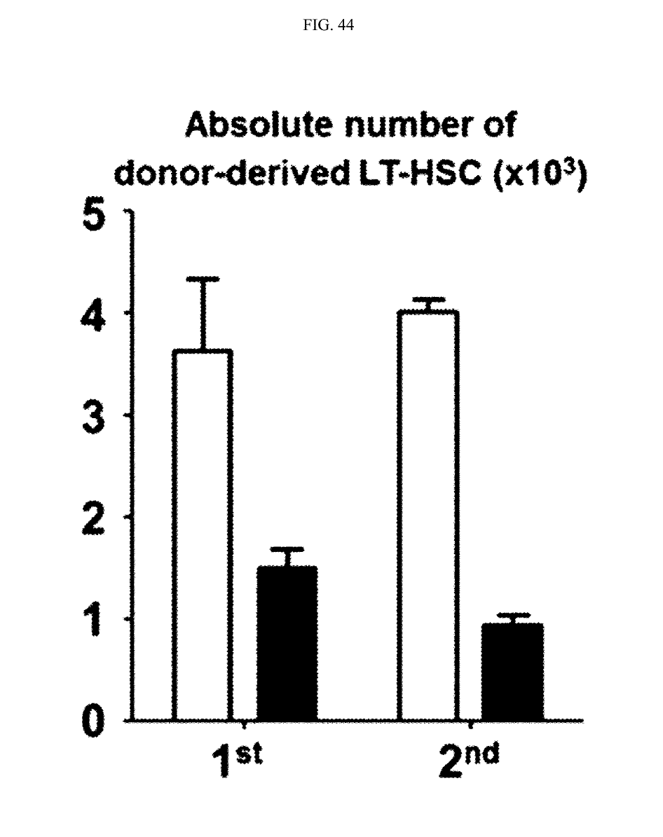

Subsequently, through serial BMT, the number of LSK cells and LT-HSCs at the second BMT were compared between BM cells of mice receiving HSPCs of the KAI1^{-/-} mice and BM cells of mice receiving HSPCs of WT mice. As a result, as shown in FIGS. 43 and 44, it can be confirmed that the number of LSK cells and LT-HSCs were lower in the BM cells of mice receiving HSPCs of the KA1^{-/-} mice than in those of the mice receiving HSPCs of WT mice.

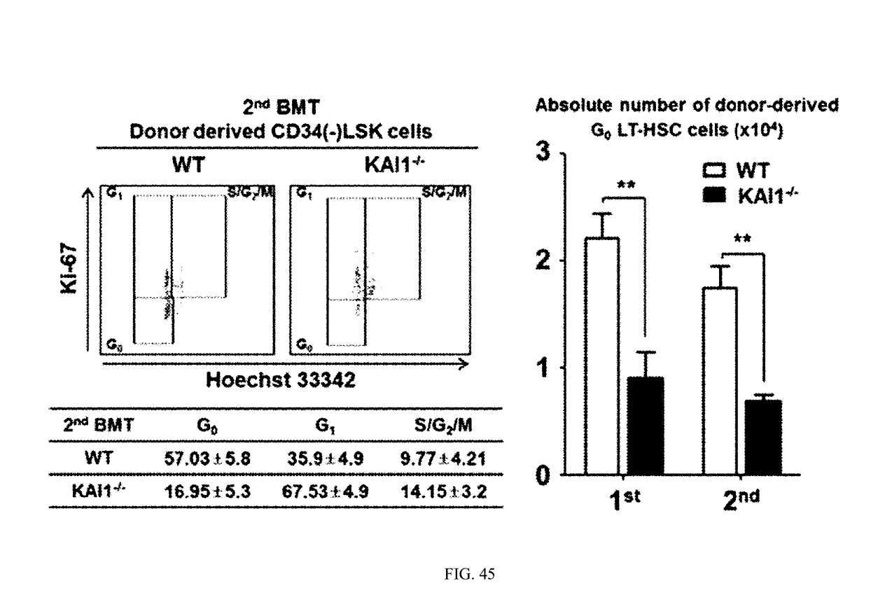

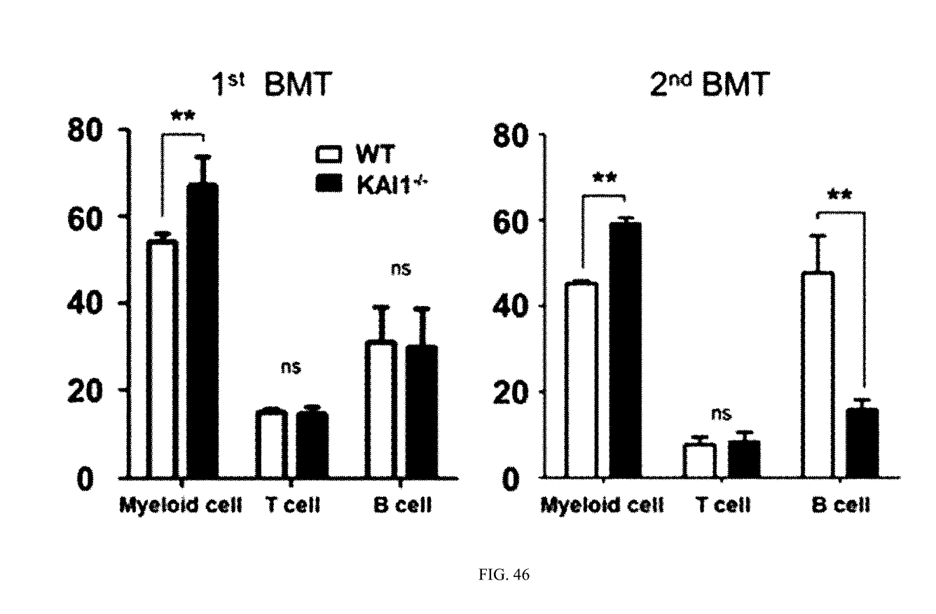

Afterwards, cell cycles in CD34− LSK cells of the mice receiving either KAI1^{-/-} mouse- or WT-derived HSPCs were compared. As a result, as shown in FIG. 45, it can be confirmed that the number and degree of G0 phase LT-HSCs were higher in the mice receiving the WT-derived HSPCs. In addition, as shown in FIG. 46, it can be confirmed that a differentiation direction of LT-HSCs went towards a myeloid lineage in the mice receiving the KAI1^{-/-} mouse-derived HSPCs.

### Example 7

### Identification of KAI1 Activity-Associated Ligand

First, to find a ligand increasing KAI1 activity, immunoprecipitation on EML cells was carried out using a KAI1 antibody, and then Western blotting was carried out using a

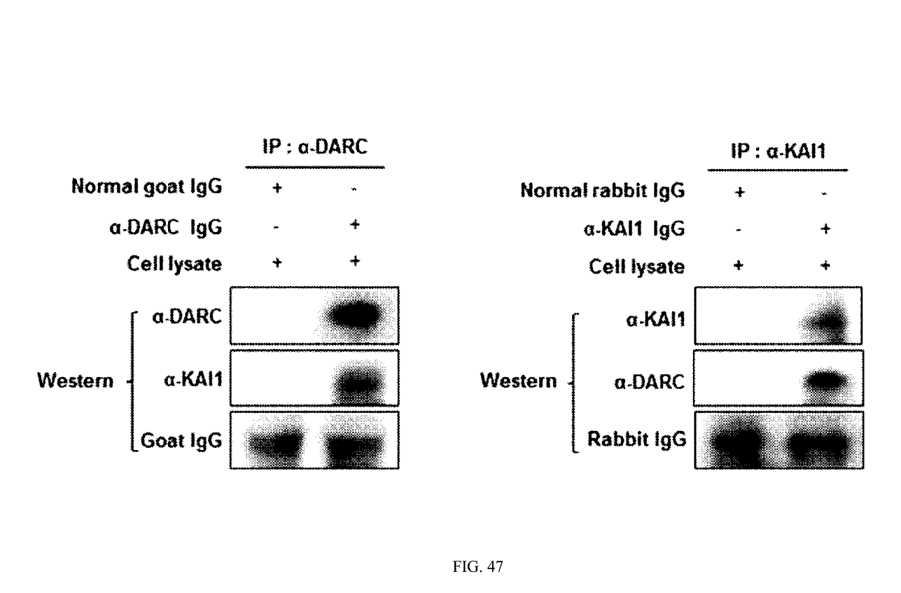

Duffy antigen receptor for chemokines (DARC). As a result, as shown in FIG. 47, it can be confirmed that KAI1 was able to interact with DARC. The same result was obtained from the immunoprecipitation (IP) of DARC and Western blotting of KAI1.

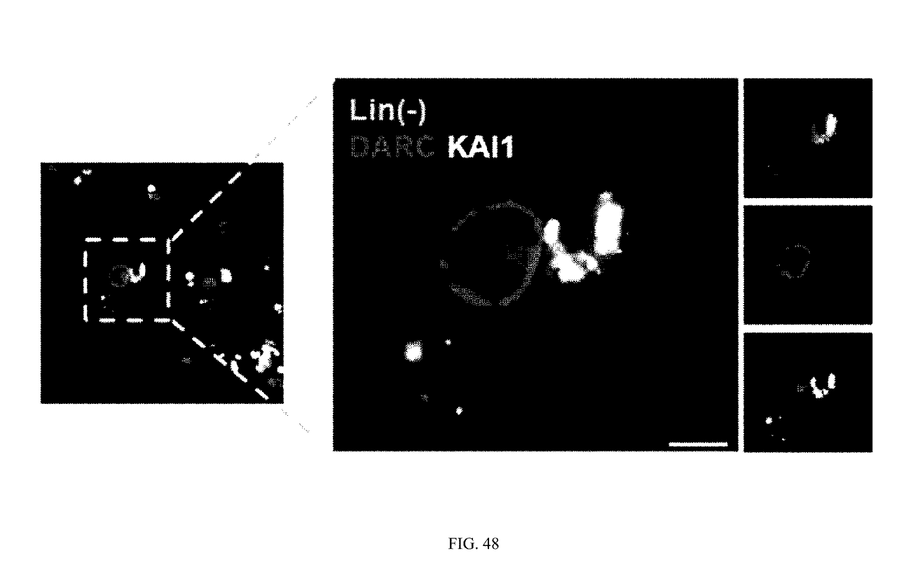

Subsequently, to show in vitro interaction between KAI1 and DARC, F4/80 positive cells were sorted from the bone of a C57 mouse using a MACS technique and seeded on a dish, and then an HSC enriched population of a C57 mouse, that is, Lin(−) cells of the C57 mouse, was sorted using a MACS technique and then tagged with CFSE. These two types of cells were co-cultured and then identified by staining with KAI1 and DARC. Therethrough, as shown in FIG. 48, it can be confirmed that KAI1 derived from HSCs and DARC derived from macrophages interacted with each other.

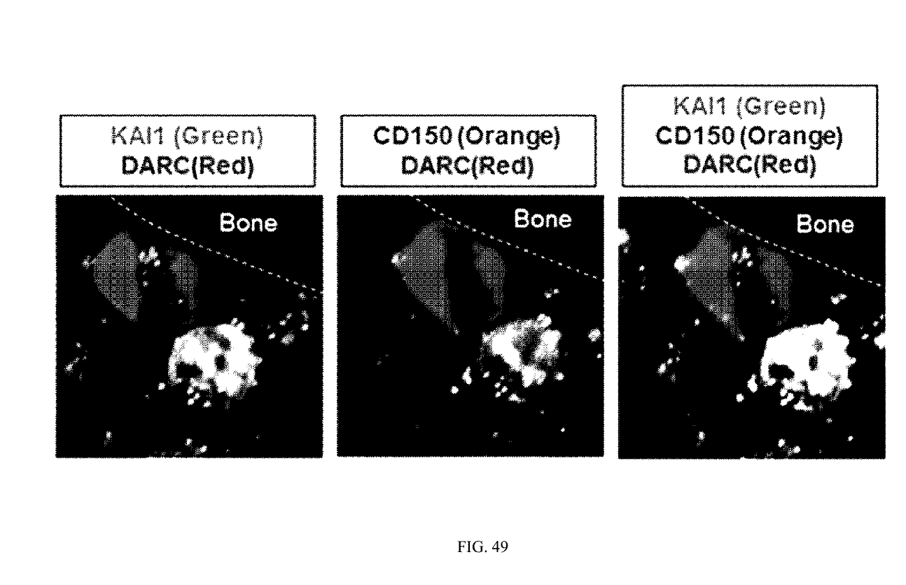

Afterwards, to confirm that KAI1 and DARC actually interacted with each other in vivo, a femur of the C57 mouse was fixed and decalcificated, followed by preparation of a paraffin block. After preparing sections, the resulting sections were subjected to deparaffinization and retrieval, and then fluorescence-stained using an antibody. As a result of staining with CD150 as a HSC marker, and as a result of KAIland DARC, it can be confirmed that KAI1(+)LT-HSC and DARC interacted with each other, as shown in FIG. 49A.

In addition, a femur of a Tie2-GFP mouse expressing GFp only in cells expressing Tie2 was treated using the above-described method for preparing a paraffin block, preparing sections, and following with deparaffinization and retrieval for fluorescence staining. Here, Tie2 was LT-HSCs. As a result, as shown in FIG. 49B, it can be confirmed that KAI1(+)LT-HSC and DARC actually interacted with each other. From the above results, it can be seen that KAI1 derived from LT-HSC interacted with DARC.

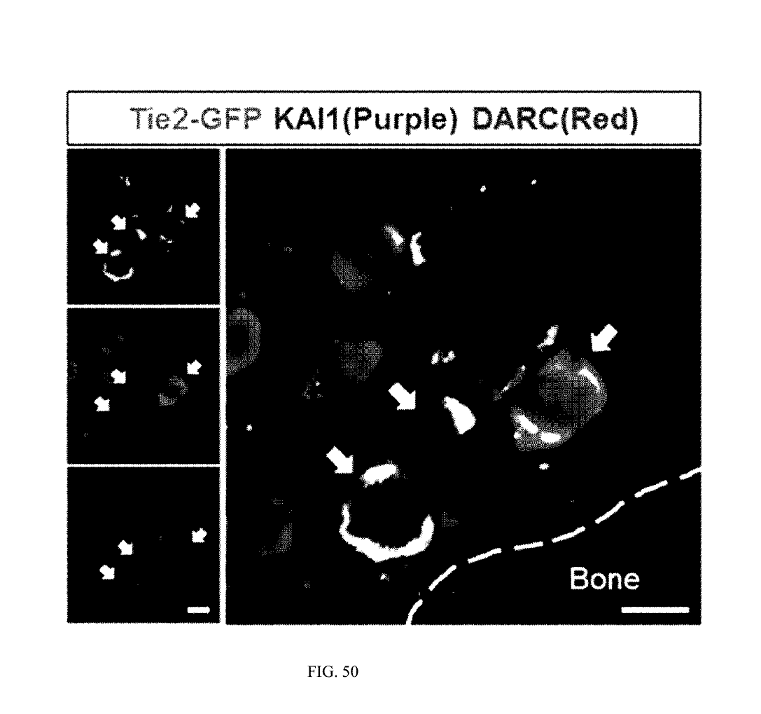

Then, to identify DARC(+) cells in BM, DARC expression was confirmed in endothelial cells, stromal cells, and monocytes/macrophages known to support HSCs, particularly in BM. Endothelial cells were identified from CD45(−)Ter119(−) CD31(+)PDGFRa(−), stromal cells were identified from CD45(−)Ter119(−)CD31(-) PDGFRa(+), and finally macrophages were identified from F4/80(+). As a result, as shown in FIG. 50, it can be confirmed that the endothelial cells and the stromal cells were mostly DARC negative cells, and the macrophages were mostly DARC positive cells.

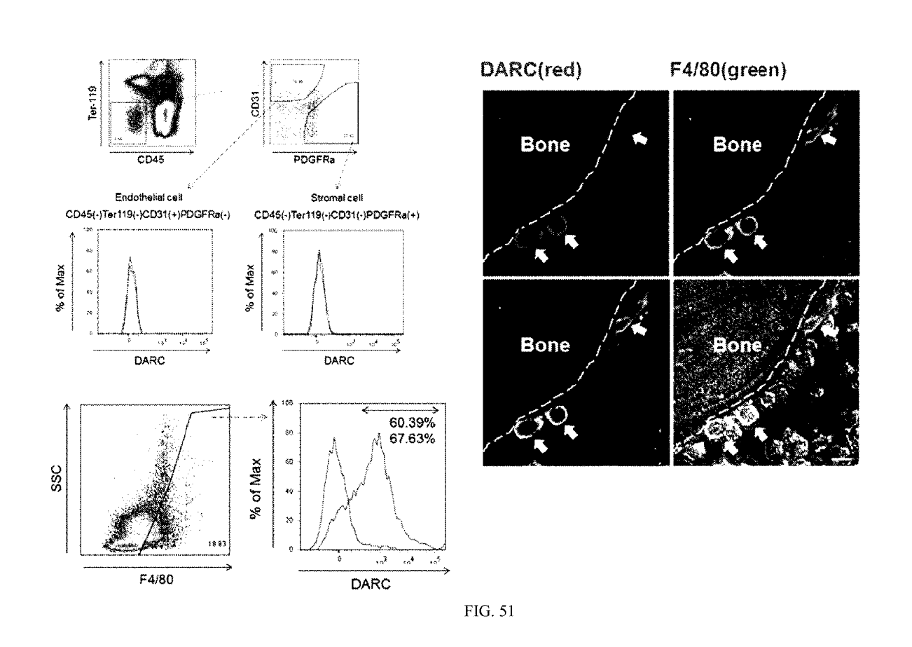

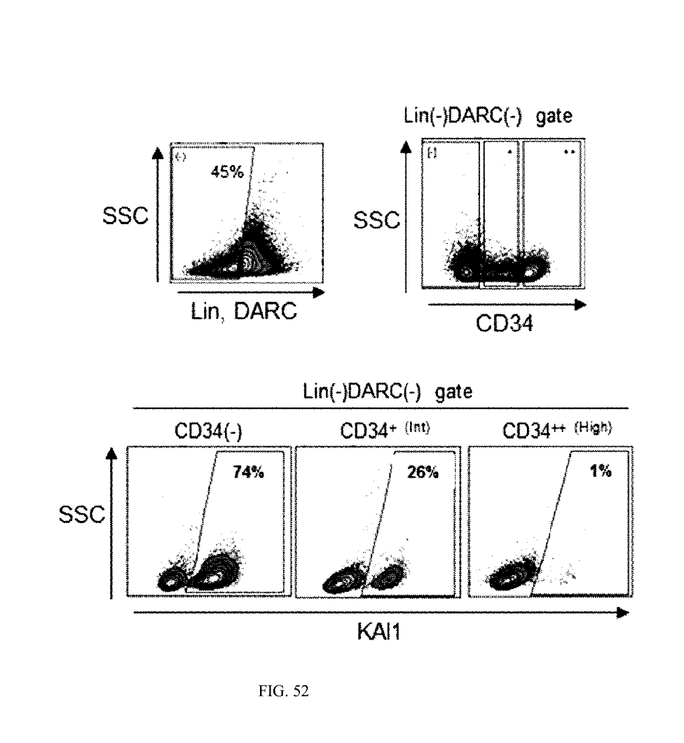

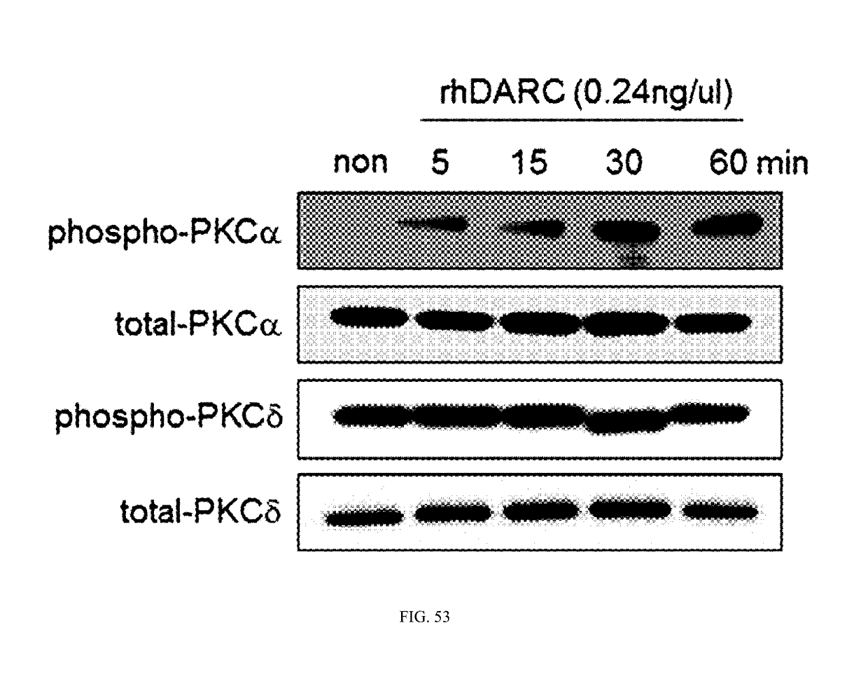

Meanwhile, in Lin(−)CD34(−), known as a quiescent cell population of EML cells, the percentage of KAI1 was approximately 80%. However, among the cell cycle-active population, Lin(−)CD34(+) cells exhibited a percentage of KAI1 of approximately 1%, which were mostly KAI1 negative cells (refer to FIG. 51). Here, to see an increase in KAI1 activity by recombinant DARC, Lin(−)DARC(−)CD34(−) cells, most of which were KAI1(+) EML cells, were sorted using a MACS technique. Specifically, EML cells were incubated with a lineage cocktail, DARC, and a CD34 primary antibody for 30 minutes at 4° C., and then washed three times. The resultant cells were treated and incubated with a secondary antibody suitable for the primary antibody for 30 minutes at 4° C. and washed three times, thereby obtaining a Lin(−)DARC(−)CD34(−) quiescent EML cell population (qEML) using a magnetic column. The cells obtained thereby were subjected to starvation for 1 to 3 hours in an almost stem cell factor (FSC)-free, 1% FBS-containing IMDM medium for 3 hours, and then treated with rhDARC (recombinant human DARC) to confirm PKC phosphorylation by KAI1 activity. As a result, as shown in FIG. 52, it can be confirmed that PKCa activity was increased by rhDARC. From the result, it was seen that KAI1 can be activated by rhDARC. DARC has approximately 76% homology with a mouse and a human, and the rhDARC acts on a mouse. In addition, to see if rhDARC increases PKCa phosphorylation using KAI1, EML cells were transfected with a Mock shRNA lentivirus and a KAI1 shRNA lentivirus [KAI1 knock-down], and then Lin(−)CD34(−)DARC(−) quiescent EML cells were isolated and treated with rhDARC. As a result, as shown in FIG. 53, it was confirmed through KAI1 that rhDARC increased PKCa phosphorylation.

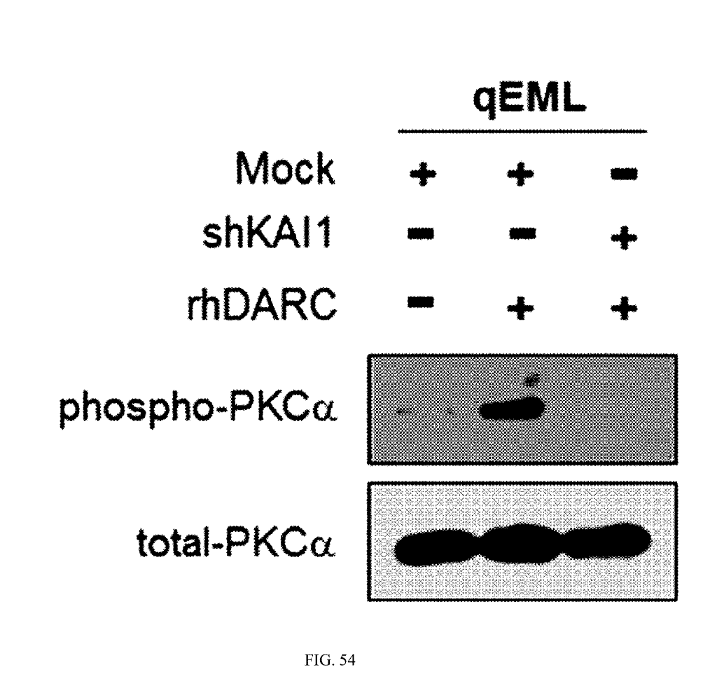

Afterwards, to confirm that G0 phase was actually maintained in a qEML cell cycle by rhDARC or co-culture of monocytes in the qEML cells from which the previously mentioned Lin(−)CD34(−)DARC(−) EML cells were sorted, the cell cycle was analyzed. More specifically, a cell cycle was analyzed by co-culturing Mock raw 264.7 cells, DARC knock-down raw 264.7 cells, and finally, DARC knock-down raw 264.7 cells with qEML cells, and then incubating the resulting cells for 2 days after pretreating with rhDARC. Therethrough, as shown in FIG. 54, it can be confirmed that, when qEML cells were co-cultured with Raw 264.7 cells, G0 cells were maintained at a high level of approximately 61%, and when co-cultured with DARC Knock-down Raw 264.7 cells, G0 phase was reduced. Here, it was confirmed that the effects were reversed by treatment with rhDARC. Consequently, it was confirmed through mono/macrophage DARC that quiescence was maintained due to KAI1 of the qEML cells.

### Example 8

### Confirmation of Mechanism of Regulating KAI1 Expression in KAI1(+)LT-HSC

KAI1(+)LT-HSC had a resistance to stress, mobilization, and emergency niches, and in a subsequent recovery process, KAI1 expression was reduced, proliferated, or differentiated in LT-HSC. Therefore, the KAI1 expression was increased or decreased by mechanism of the LT-HSC.

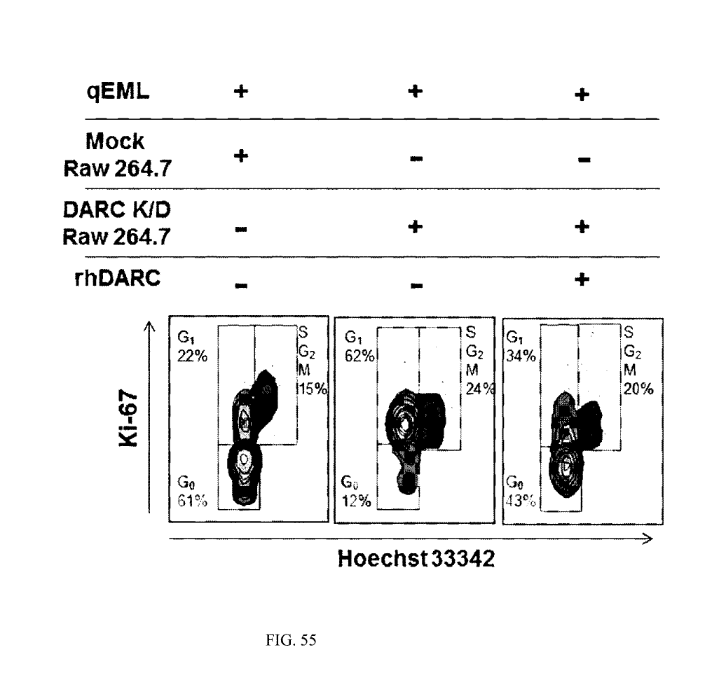

First, EML cells were mixed with various types of cells. Lin(−)CD34(−) cells were sorted using MACS, and cultured with a stem cell factor (SCF) to identify KAI1 (+) cells, and therethrough, as shown in FIG. 55, it was confirmed that the KAI1(+) cells were reduced to 12% within two days. In addition, when the KAI1(+) cells were identified after co-culture with a mouse macrophage cell line, that is, Raw 264.7 cells, as shown in FIG. 55, it can be confirmed that KAI1-expressed cells in quiescent EML cells were maintained at a level of 83%, similar to that after MACS sorting, through the co-culture with the Raw 264.7 cells. From the above result, it can be seen that KAI1 expression in LT-HSC was maintained by macrophages, which have a direct effect and a paracrine effect.

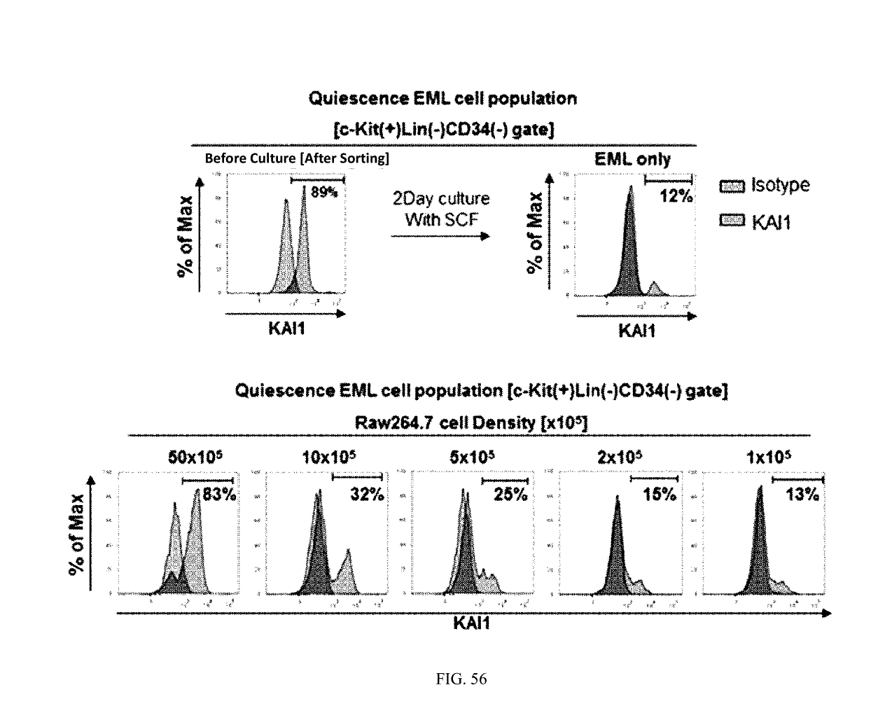

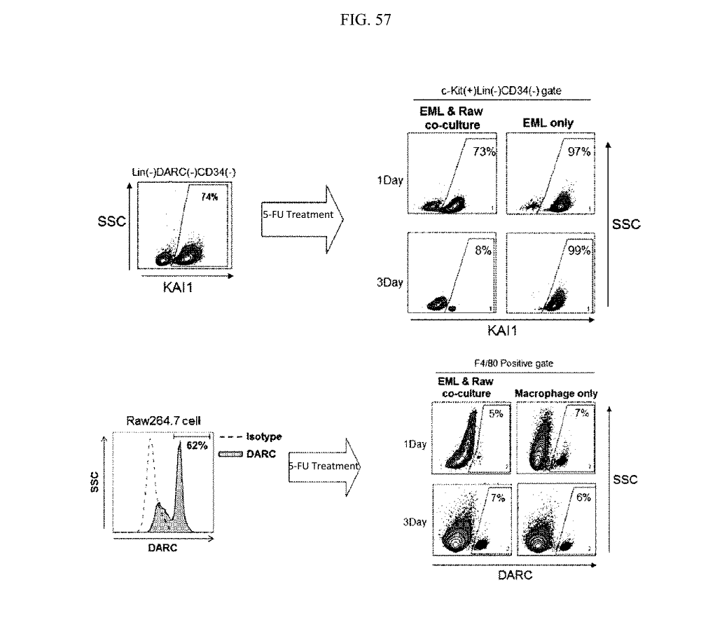

Then, when DARC expression in the Raw 264.7 cells was confirmed at a stress or mobilization niche such as G-CSF or 5-FU, as shown in FIGS. 56 and 57, it was confirmed that DARC expression was decreased at the stress or mobilization niche such as G-CSF or 5-FU, and KAI1 of LT-HSC was also reduced. From the result, it can be seen that the reduction in KAI1 of LT-HSC was caused by a direct or indirect effect of DARC.

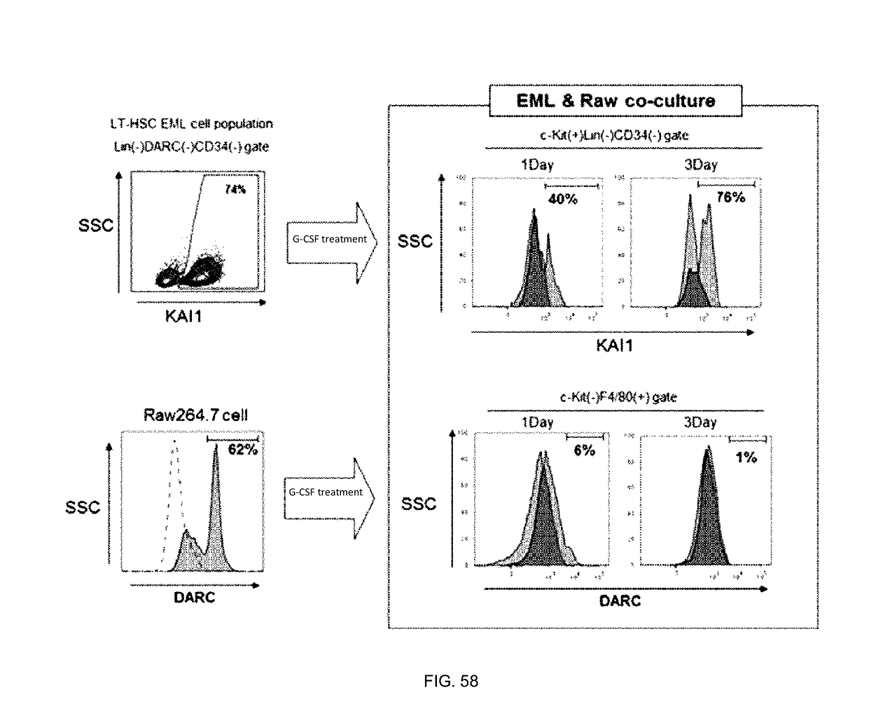

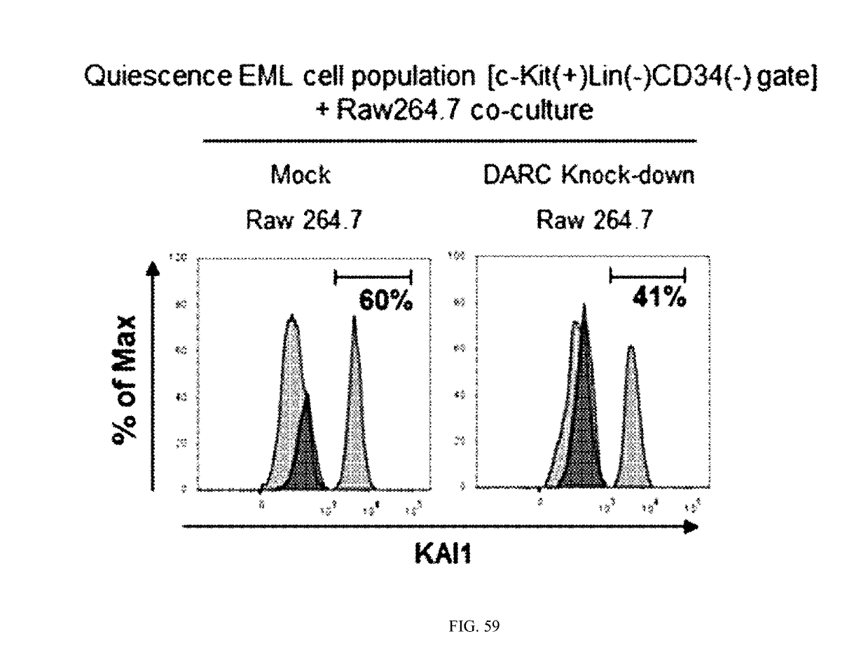

Afterwards, after the DARC expression in Raw 264.7 cells was reduced using shRNA, quiescent EML cells sorted in the same manner as described above from total EML cells and co-cultured with Mock Raw 264.7 and DARC Knock-down Raw264.7 cells under the condition of SCF addition. As a result, as shown in FIG. 58, it can be confirmed that KAI1 expression in the quiescent EML cells was reduced by co-culture with the DARC Knock-down Raw 264.7 cells. In addition, to see a paracrine effect, Mock and DARC Knock-down Raw 264.7 supernatants were obtained and cultured with EML cells. Consequently, as shown in FIG. 59, it can be confirmed that KAI1 expression was maintained even by a paracrine effect, and KAI1 expression in the quiescent EML cells was reduced in a supernatant obtained from the DARC Knock-down Raw 264.7 cells.

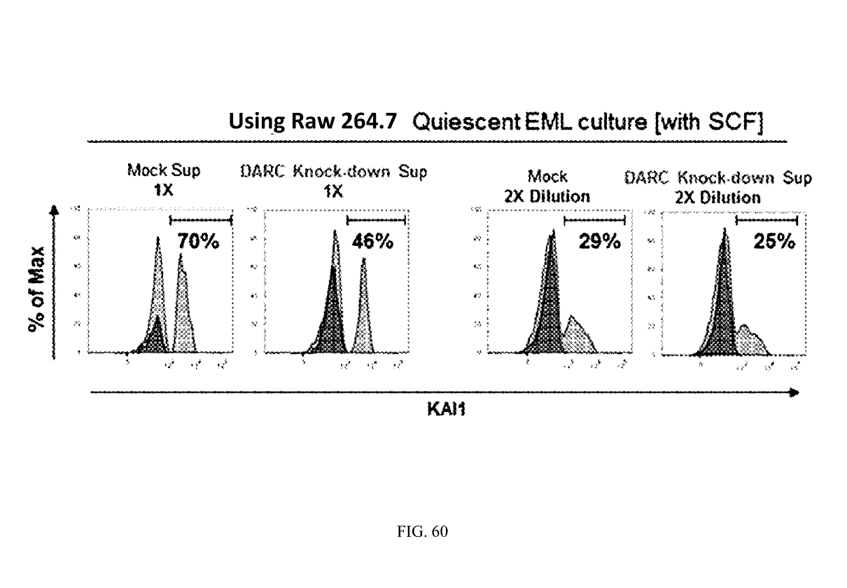

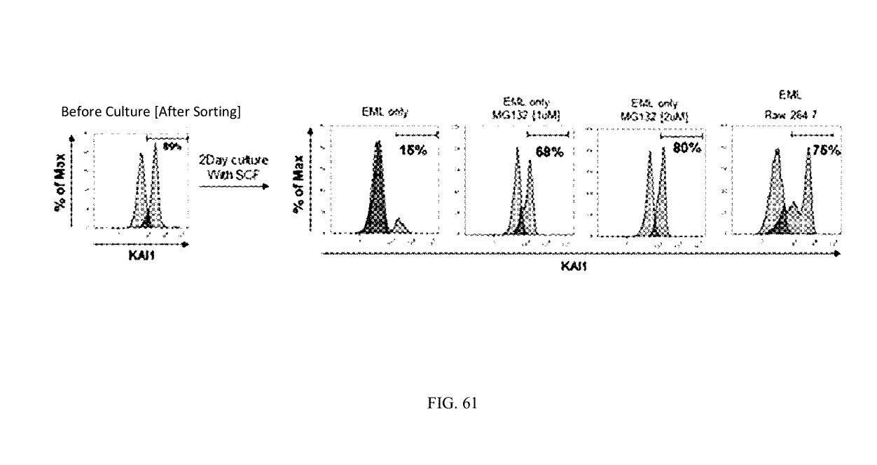

Moreover, since KAI1 was reduced by two processes such as ubiquitination and endocytosis, qEML cells were sorted from EML cells and cultured with SCF. Therethrough, as shown in FIG. 60, it can be confirmed that under the condition of addition of a ubiquitin degradation inhibitor such as MG-132, KAI1 expression was not reduced. In addition, the qEML was cultured with SCF, and after 8 hours, KAI1 was immunoprecipitated, and mono/poly-ubiquitin was detected using Western blotting, thereby confirming ubiquitinated KAI1, as shown in FIG. 61.

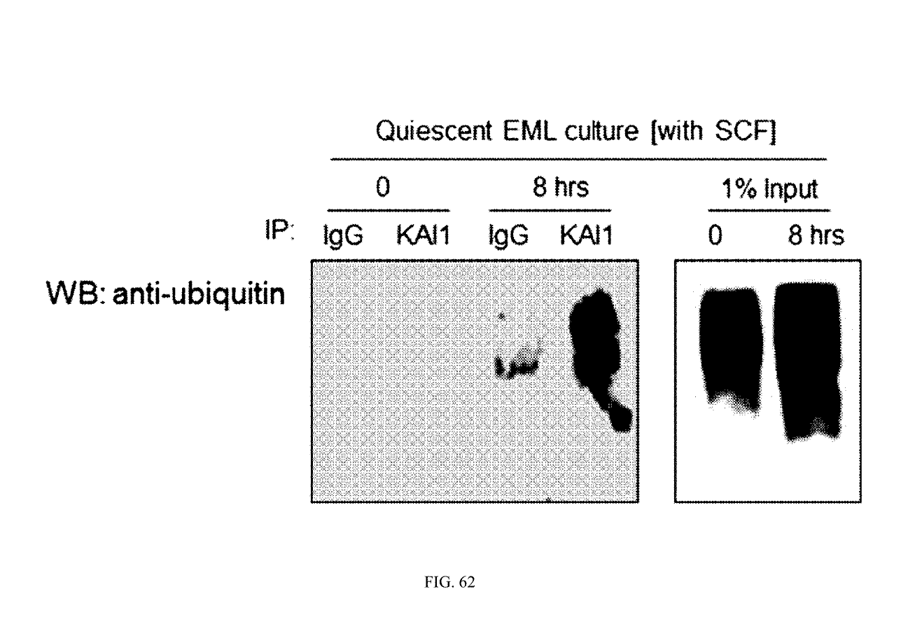

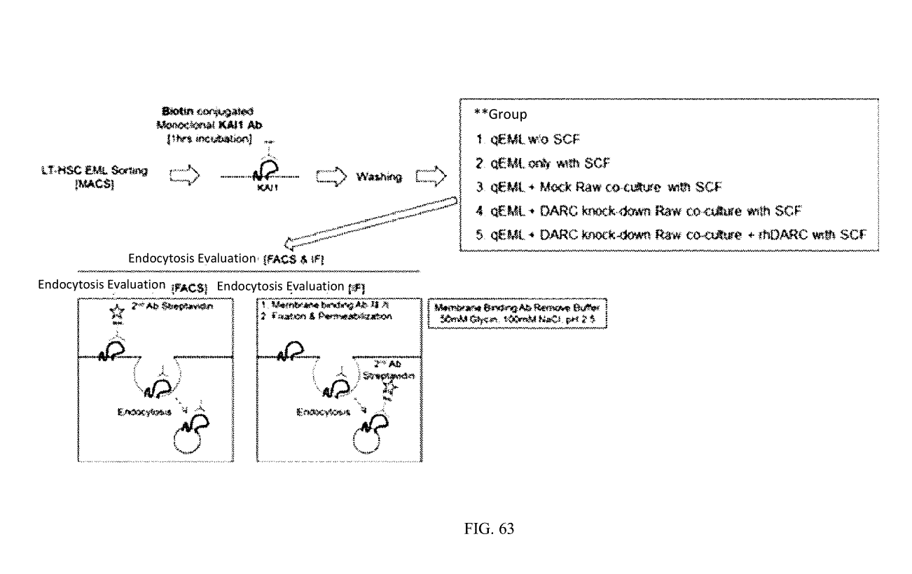

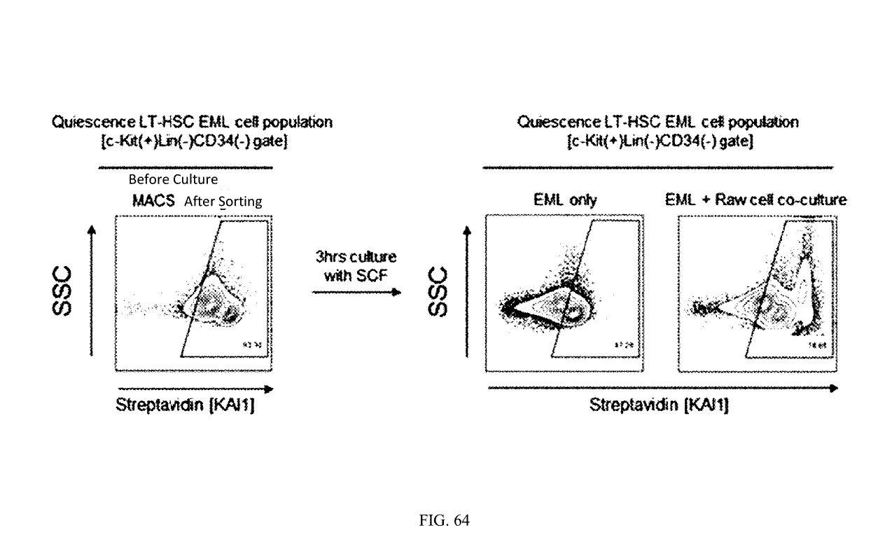

Furthermore, to evaluate endocytosis, qEML cells were obtained from EML using MACS in the same manner as described above, incubated with a biotin-conjugated KAI1 antibody at 4 □ for 1 hour, and then washed to remove a non-bound antibody. Afterwards, to identify qEML only, qEML+Mock Raw 264.7 cells, qEML+DARC Knock-down Raw 264.7, qEML+DARC Knock-down Raw 264.7+rhDARC, and finally, to confirm that the endocytosis was caused by ubiquitination, qEML cells were pretreated with MG-132. In addition, all groups were treated with SCF, cultured for 3 hours, and then had a membrane-binding antibody removed using an acidic buffer (50 mM glycine, 100 mM NaCl, pH 2.5). Moreover, to identify endocytic KAI1-biotin conjugation, a streptavidin-555 secondary antibody was used after fixation/permeabilization. As a result, as shown in FIGS. 62 to 64, [SCF] KAI1 was ubiquitinated by a growth factor when only qEML was cultured, resulting in endocytosis, and therefore KAI1 was reduced. However, when co-culturing with a monocyte/macrophage first, it can be confirmed that KAI1 endocytosis was not reduced by DARC.

### Example 9

### Confirmation of Role of KAI1(CD82) in Maintaining Quiescence of Human LT-HSC

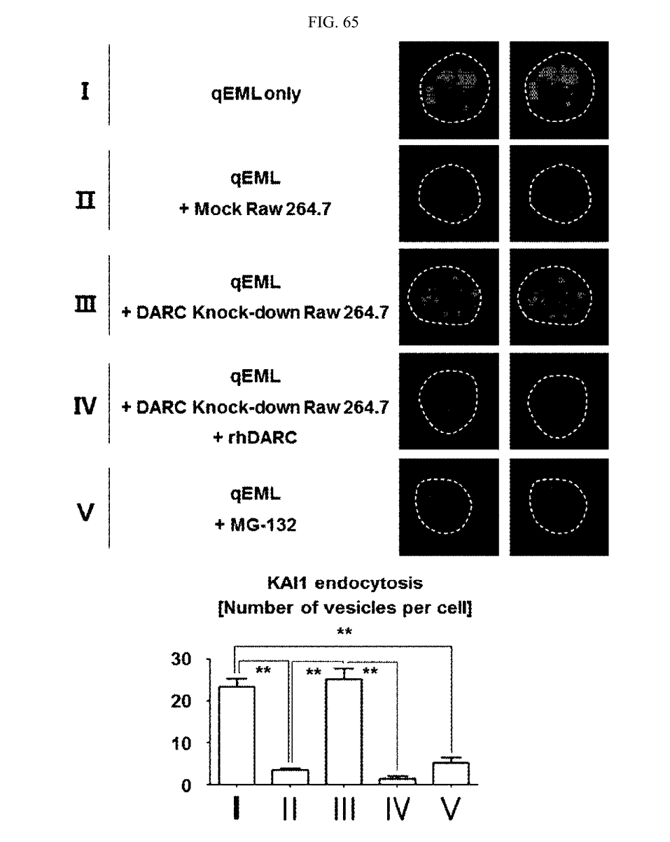

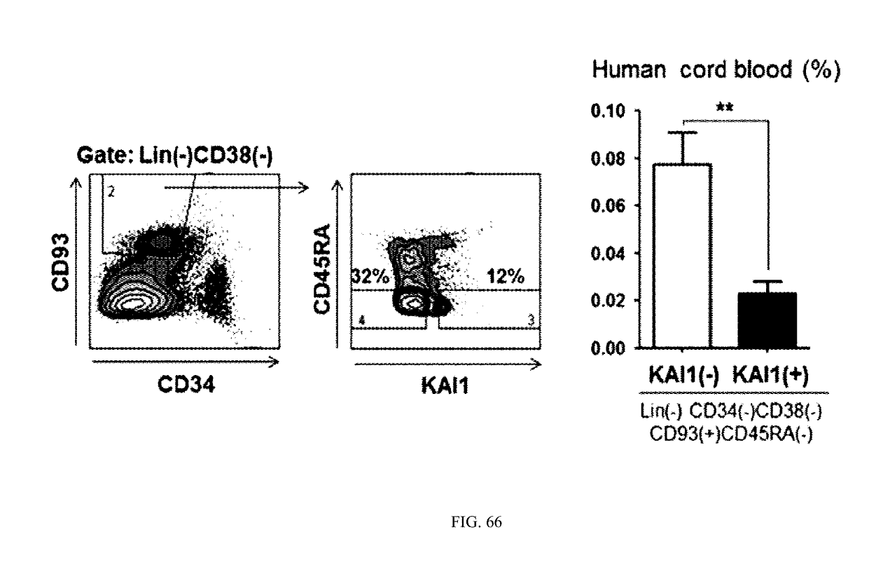

To confirm if the experimental result showing that KAI1 expression is critical for maintaining the quiescence of mouse LT-HSC is also applicable to humans, an experiment was carried out using human umbilical cord blood from a blood vessel of an umbilical cord. The umbilical cord blood was collected with a heparin-coated syringe under the consent of a donor and delivered. Monocytes contained in the blood were collected using histopaque 1077. These were stained with a LIN, CD38, CD34, CD93, or CD45RA antibody, and analyzed by FACS. As a result, as shown in FIGS. 65 and 66, it can be confirmed that from the LIN(−CD38(−)CD34(−)CD93(+)CD45RA(−) population, KAM+) LT-HSCs derived from human LT-HSCs accounted for approximately 25%, and unlike a KAI1(−) population, most of the KAI1(+) LT-HSCs were at G0 phase. From the result, it can be seen that the human LT-HSCs expressed KAI1, and KAI1-expressed human LT-HSCs were quiescent.

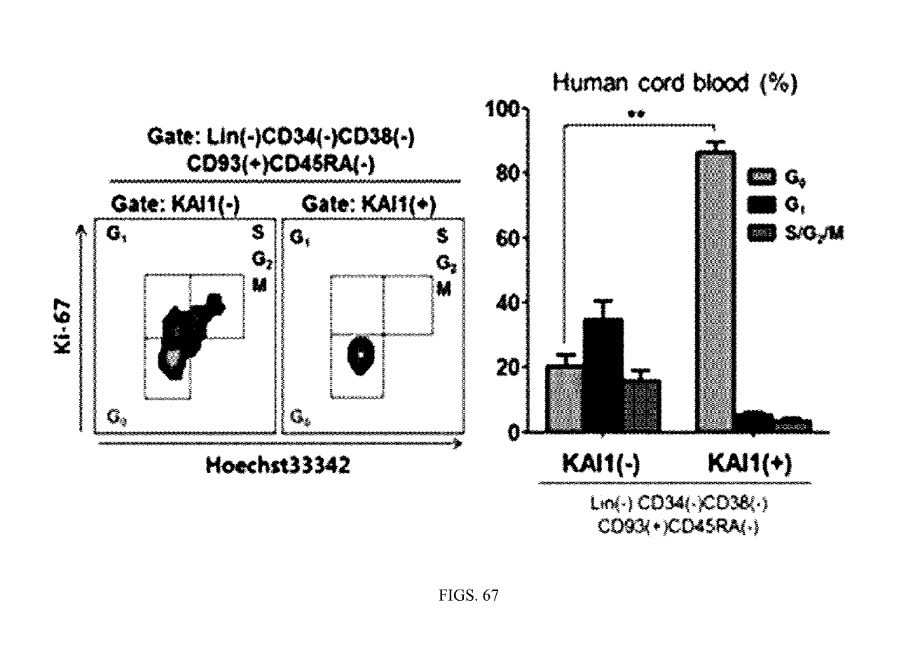

In addition, first MNC was obtained first from the human umbilical cord blood and Lin(+)CD235(+)CD235(+) cells were removed using MACS, and then KAI1(+)LT-HSC and KAI1(−)LT-HSC were sorted using FACS. Then, following 3-hour starvation, the cells were treated with rhDARC for 30 minutes, and subjected to fixation/permeabilization and fluorescence staining to confirm PKCa phosphorylation. As a result, as shown in FIG. 67, it can be confirmed that, in humans, PKCa phosphorylation was increased in the KAI1(+)LT-HSC by rhDARC.

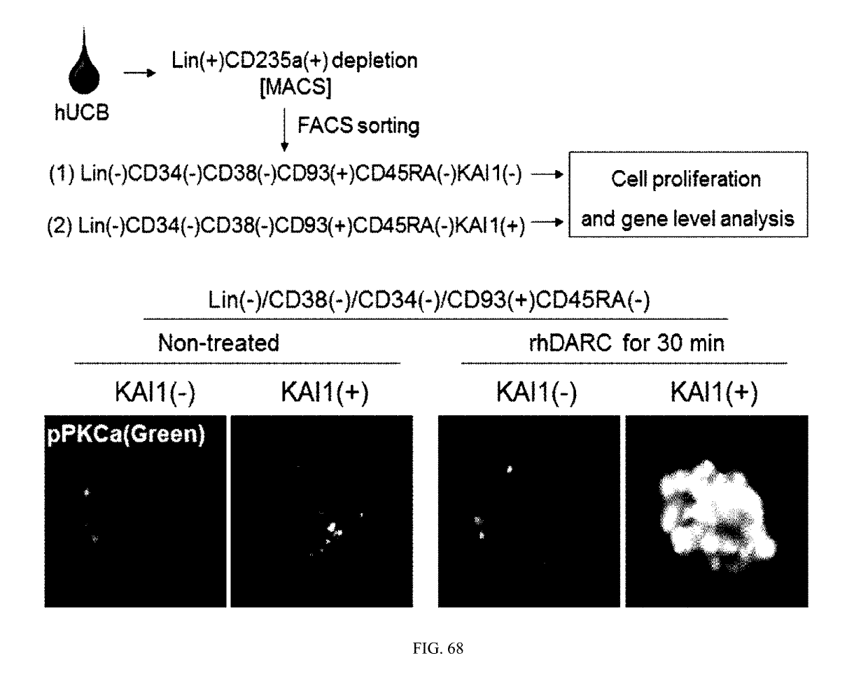

Afterwards, as an experiment to confirm if quiescence of KAI1(+)LT-HSC was increased by rhDARC, MNC was obtained from human umbilical cord blood by the same method as described above, Lin(+)CD235(+)DARC(+) cells were removed, and following 3-hour starvation, the cells were treated with rhDARC for 2 days, and then a cell cycle was analyzed. As a result, as shown in FIG. 68, it can be confirmed that the number of cells in G0 phase was increased in KAI1(+)LT-HSC due to rhDARC.

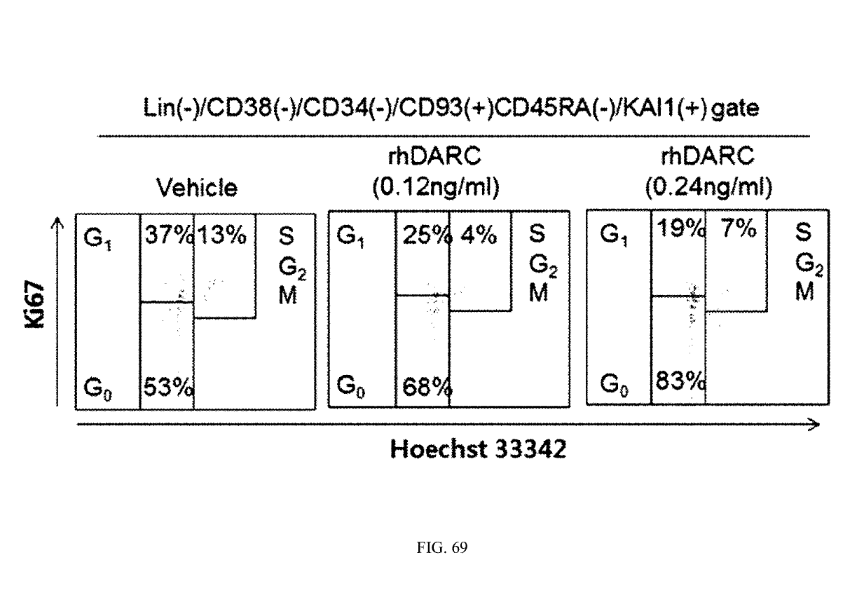

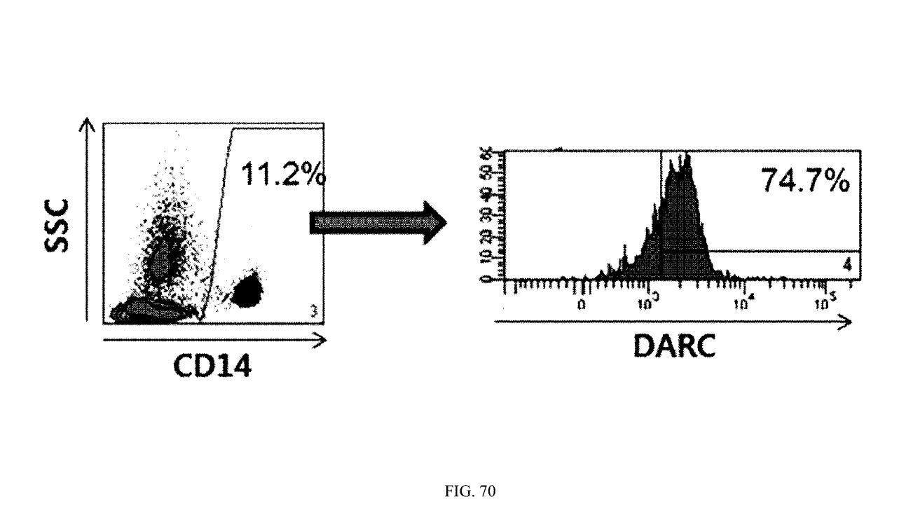

Subsequently, according to the experiment subjected for a human carried out in the same manner as described above, as shown in FIG. 69, it can be confirmed that in cord blood, most of CD14 positive cells as a monocyte/macrophage marker were DARC positive cells.

Finally, to confirm if KAI1 expression in LT-HSC was also regulated by monocytes in humans, Lin(−)CD235(−)DARC(−) cells were obtained from umbilical cord blood by the same method as described above and some of those cells were isolated as monocytes [un-touched monocytes] using MACS. According to a [SCF, Flt3-ligand, TPO, IL-3] HSC-only culture obtained using a growth factor, it can be confirmed that, as shown in FIG. 70, KAI1 was reduced, but KAI1 expression was maintained by the co-culture with a monocyte or rhDARC without reduction.

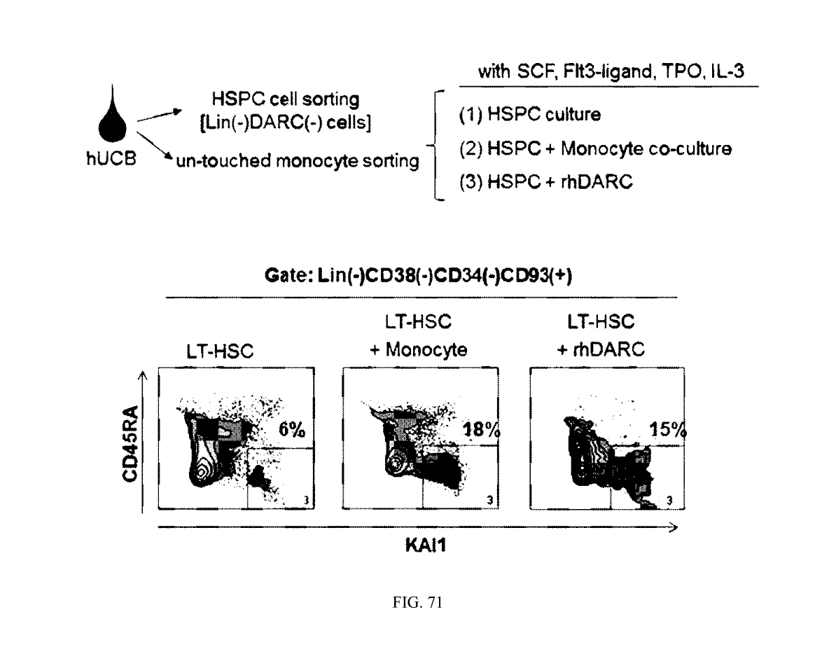

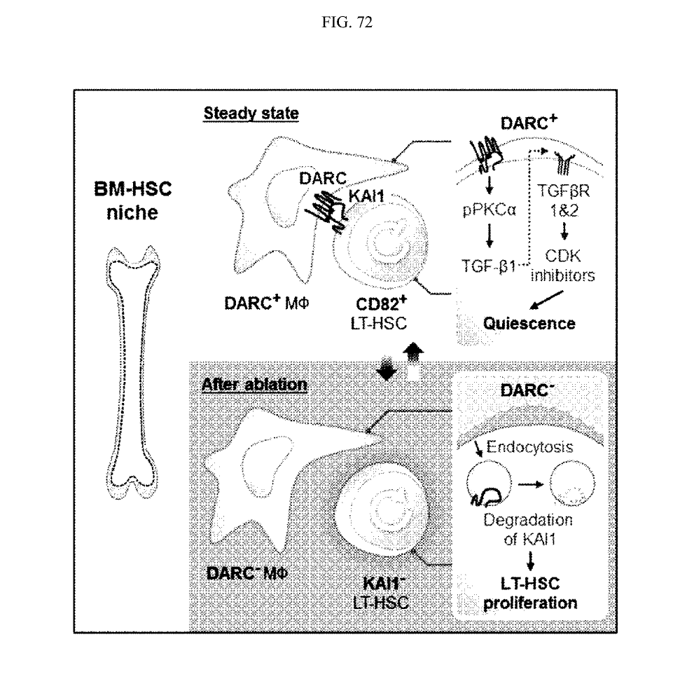

The above-mentioned results are summarized and shown in FIG. 71. As shown in FIG. 71, first, KAI1 of LT-HSC was stimulated by DARC of monocytes/macrophages in homeostasis [increase in KAI1 phosphorylation] to phosphorylate PKCa, increase TGFb, and increase CDK inhibitors such as p21, p27, and p57 via Smad2/3 through TGFR1&2, thereby reducing phospho-Rb and resulting in maintaining the quiescence of the LT-HSC. Second, in a situation involving an emergency/mobilization/stressed niche such as 5-FU or G-CSF, due to the reduction in expression of the DARC of the monocytes/macrophages or the number of monocytes/macrophages, a KAI1/DARC complex was broken, and thereby the KAI1 of the LT-HSC was ubiquitinated and subjected to endocytosis by a neighboring growth factor. Therefore, the cell cycle and differentiation of KAI1(−) LT-HSC were improved, resulting in bone marrow regeneration. DARC(+)monocytes/macrophages were increased again by the bone marrow regeneration. Particularly, the KA1I(−)LT-HSC was able to be proliferated and differentiate into the monocyte/macrophage. As a result, the KAI1 of the LT-HSC was increased again, and maintained homeostasis.

It would be understood by those of ordinary skill in the art that the above description of the present invention is exemplary, and the exemplary embodiments disclosed herein can be easily modified into other specific forms without departing from the technical spirit or essential features of the present invention. Therefore, the exemplary embodiments described above should be interpreted as illustrative and not limited in any aspect.

1-12. (canceled)

13. A method of purifying hematopoietic stem cells having a resistance to stress, comprising:
(a) analyzing KAI1 expression in an isolated hematopoietic stem cell population; and
(b) isolating and collecting only KAI1-expressed hematopoietic stem cells.

14. A cellular therapy product for treating blood tumor, comprising hematopoietic stem cells purified by the method of claim 13.

15. A method of collecting quiescent hematopoietic stem cells, comprising:
increasing KAI1 expression in hematopoietic stem cells, wherein the hematopoietic stem cells are long-term hematopoietic stem cells (LT-HSCs), short-term hematopoietic stem cells (ST-HSCs), or multipotent progenitors (MPPs).

16. The method of claim 15, wherein the hematopoietic stem cells are cultured with a rhDARC, DARC positive monocyte, DARC positive macrophage, or DARC positive macrophage culture solution to increase or maintain KAI1 expression.

17. Quiescent hematopoietic stem cells collected by the method of claim 15.

18. The quiescent hematopoietic stem cells of claim 17, which are enhanced in in vivo transplantation efficiency, proliferation, differentiation, storage ability, or stability.

19. The quiescent hematopoietic stem cells of claim 17, which are improved in organ transplantation or regeneration ability.

20. A method of regulating the cell cycle of hematopoietic stem cells, comprising:
treating hematopoietic stem cells with a composition comprising a KAI1(CD82) polypeptide or a gene encoding the same.

21. A method of preventing or treating blood tumor, comprising:
administering a composition comprising a KAI1(CD82) polypeptide or a gene encoding the same to a subject in need thereof.

22. (canceled)

23. The method of 21, wherein the composition further comprises a DARC polypeptide or a gene encoding the same.

24. The method of 21, wherein the KAI1 polypeptide consists of the amino acid sequence represented by SEQ ID NO: 1 or SEQ ID NO: 2.

25. The method of 21, wherein the DARC polypeptide consists of the amino acid sequence represented by SEQ ID NO: 3 or SEQ ID NO: 4.

26. The method of 21, wherein the blood tumor is selected from the group consisting of lymphoma, multiple myeloma, myelogenous leukemia, and lymphocytic leukemia.

27. The method of 20, wherein the composition increases TGF-β1 secretion via PKC.
